(19) United States
(12) Patent Application Publication
(10) Pub. No.: US 2011/0230428 A1
(43) Pub. Date: Sep. 22, 2011

(54) CERTAIN KYNURENINE-3-MONOOXYGENASE INHIBITORS, PHARMACEUTICAL COMPOSITIONS, AND METHODS OF USE THEREOF

(76) Inventors: John Wityak, Carlsbad, CA (US); Leticia M. Toledo-sherman, Santa Monica, CA (US); Celia Dominguez, Los Angeles, CA (US); Stephen Martin Courtney, Oxfordshire (GB); Christopher John Yarnold, Oxfordshire (GB); Paula C. De Aguiar Pena, Oxfordshire (GB); Andreas Scheel, Hamburg (DE); Dirk Winkler, Hamburg (DE)

(21) Appl. No.: 13/054,931

(22) PCT Filed: Jul. 22, 2009
(86) PCT No.: PCT/US09/04244

§ 371 Date: Apr. 13, 2011

(30) Foreign Application Priority Data
Jul. 22, 2008 (US) 61/082744

(51) Int. Cl. A61K 31/7024; C07D 271/06; A61K 31/4245; C07D 233/64; A61K 31/4164; C07D 257/04; A61K 31/41; C07C 251/38; A61K 31/15; C07D 207/50; A61K 31/40; C07C 255/64; A61K 31/277; C07D 471/04; A61K 31/437; C07C 311/05; A61K 31/18; A61K 31/192; C07D 295/13; A61K 31/5375; C07H 13/04; A61P 25/28; A61P 25/16; A61P 25/00; A61P 25/08

(52) U.S. Cl. 514/23; 548/131; 514/364; 548/336.1; 514/399; 548/252; 514/381; 564/256; 514/640; 548/557; 514/426; 558/408; 514/523; 546/118; 514/303; 564/99; 514/605; 562/440; 514/567; 544/168; 514/238.2; 514/237.5; 544/162; 536/18.7

(57) ABSTRACT

Certain chemical entities are provided herein. Pharmaceutical compositions comprising at least one chemical entity and one or more pharmaceutically acceptable vehicle. Methods of treating patients suffering from certain diseases and disorders responsive to the inhibition of KMO activity are described, which comprise administering to such patients an amount of at least one chemical entity effective to reduce signs or symptoms of the disease or disorder are disclosed. These diseases include neurodegenerative disorders such as Huntington's disease. Methods of treatment include administering at least one chemical entity as a single active agent or administering at least one chemical entity in combination with one or more other therapeutic agents. Also provided are methods for screening compounds capable of inhibiting KMO activity.

This application claims priority to U.S. Provisional Application No. 61/082,744, filed Jul. 22, 2008, which is incorporated herein by reference.

Provided herein are certain kynurenine-3-monooxygenase inhibitors, pharmaceutical compositions thereof, and methods of their use.

Kynurenine-3-monooxygenase (KMO) is an enzyme in the tryptophan degradation pathway that catalyzes the conversion of kynurenine into 3-hydroxykynurenine (3-HK), which is a precursor of the neurotoxin quinolinic acid (QUIN). Therefore, compounds which act as inhibitors of KMO are of particular interest since they may block the metabolism toward QUIN and at the same time, may increase the formation of neuroprotective metabolite kynurenic acid (KYNA).

KMO inhibitors have been proposed as therapeutic agents for the treatment of neurodegenerative disease such Huntington's disease, Alzheimer's disease, dementia caused by Acquired Immunodeficiency Syndrome (AIDS), infarctual dementia, cerebral ischemia, cerebral hypoxia, Parkinson's disease, epilepsy, head and spinal cord injury, amyotrophic lateral sclerosis, glaucoma retinopathy, infections of the brain or inflammations of the brain. There remains a need for compounds that are effective inhibitors of KMO and may be used in treating neurodegenerative disorders.

Provided is at least one chemical entity chosen from compounds of formula I

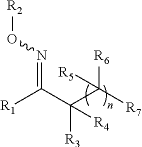

and pharmaceutically acceptable salts and prodrugs thereof wherein:
R₁ is chosen from aryl and heteroaryl, each of which is substituted with one, two, or three groups chosen from halo, lower alkyl, alkoxy, and hydroxy;
R₂ is chosen from hydrogen and optionally substituted lower alkyl;
R₃ and R₄ are independently chosen from hydrogen, halo, hydroxy, lower alkyl, and lower alkoxy;
for each occurrence, R₅ and R₆ are independently chosen from hydrogen and lower alkyl;
or R₃ and R₅, taken together with the atoms to which they are attached, form an optionally substituted cycloalkyl ring,
n is one or two;
R₇ is chosen from —C(O)OR₈, —C(O)R₈, optionally substituted amino, —C(═N—OR₁₁)R₈, —C(O)NR₉R₁₀, cyano, and optionally substituted heteroaryl;
R₈ is chosen from hydrogen, optionally substituted lower alkyl, optionally substituted aryl, optionally substituted heteroaryl, optionally substituted cycloalkyl, optionally substituted heterocycloalkyl, and glycosyl; and
R₉ and R₁₀ are independently chosen from hydrogen, optionally substituted lower alkyl, optionally substituted lower alkoxy, optionally substituted cycloalkyl, optionally substituted aryl, optionally substituted heterocycloalkyl, and optionally substituted heteroaryl;
or R₉ and R₁₀, together with the nitrogen to which they are bound, form an optionally substituted heterocycloalkyl or optionally substituted heteroaryl ring;
R₁₁ is chosen from hydrogen and optionally substituted lower alkyl,
provided that if R₁ is 4-bromophenyl, 4-fluorophenyl, 4-methoxyphenyl, 4-methylphenyl, 3-methoxyphenyl, 3-chloro-4-n-propoxyphenyl, 3-chloro-4-ethoxyphenyl, 3-chloro-4-n-butoxyphenyl, 2,3,4-trimethylphenyl, 3,4,5-trimethylphenyl, 3-chloro-4-methoxyphenyl, 2,4-dimethoxyphenyl, or 2-hydroxy-5-methylphenyl;
   n is 1;
   R₂, R₃, R₄, R₅, and R₆ are hydrogen; and
   R₇ is —C(O)OR₈, then R₈ is not methyl, ethyl, or hydrogen;
provided that if R₁ is 4-fluorophenyl;
   n is 1;
   R₂, R₃, R₄ and R₅ are hydrogen;
   R₆ is methyl; and
   R₇ is —C(O)OR₈, then R₈ is not ethyl;
provided that if R₁ is 4-chlorophenyl;
   n is 1;
   R₂, R₃, R₄ and R₅ are hydrogen and R₆ is methyl, ethyl, or butyl; or
   R₂, R₃, and R₄ and R₅ and R₆ are ethyl; and
   R₇ is —C(O)OR₈, then R₈ is not ethyl;
provided that if R₁ is 5-bromo-2-methoxyphenyl;
   n is 2;
   R₂, R₃, R₄, R₅, and R₆ are hydrogen; and
   R₇ is —C(O)OR₈, then R₈ is not methyl or ethyl;
provided that if R₁ is 2-hydroxyphenyl, 4-hydroxyphenyl, 2,4-dihydroxy-6-methylphenyl, 4-ethoxyphenyl, or 4-methoxyphenyl;
   n is 2;
   R₂, R₃, R₄, R₅, and R₆ are each hydrogen; and
   R₇ is —C(O)OR₈, then R₈ is not hydrogen;
provided that if R₁ is 3-chloro-4-methoxyphenyl or 2,5-dimethylphenyl;
   n is 1;
   R₂, R₃, R₄, and R₅ are hydrogen;
   R₆ is ethyl or propyl; and
   R₇ is —C(O)OR₈, then R₈ is not hydrogen;
provided that if R₁ is 4-methylphenyl;
   n is 1;
   R₂, R₃, R₄ and R₅ are hydrogen;
   R₆ is methyl; and
   R₇ is —C(O)OR₈, then R₈ is not hydrogen; and
provided that if R₁ is 4-methoxyphenyl;
   n is 1;
   R₂, R₄, R₅ and R₆ are hydrogen;
   R₃ is methyl; and
   R₇ is —C(O)OR₈, then R₈ is not hydrogen.

Also provided is a pharmaceutical composition comprising at least one chemical entity described herein and at least one pharmaceutically acceptable excipient.

Also provided is a packaged pharmaceutical composition comprising at least one pharmaceutical composition described herein and instructions for using the composition to treat a subject suffering from a condition or disorder mediated by Kynurenine 3-mono-oxygenase activity.

Also provided is a method of treating a condition or disorder mediated by Kynurenine 3-mono-oxygenase activity in a subject in need of such a treatment which method comprises administering to the subject a therapeutically effective amount of at least one chemical entity described herein.

As used in the present specification, the following words, phrases and symbols are generally intended to have the meanings as set forth below, except to the extent that the context in which they are used indicates otherwise. The following abbreviations and terms have the indicated meanings throughout:

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group. When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having 1 to 4 carbons.

“Cycloalkyl” indicates a saturated hydrocarbon ring group, having the specified number of carbon atoms, usually from 3 to 7 ring carbon atoms. Examples of cycloalkyl groups include cyclopropyl, cyclobutyl, cyclopentyl, and cyclohexyl as well as bridged and caged saturated ring groups such as norbornane.

By “alkoxy” is meant an alkyl group of the indicated number of carbon atoms attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Alkoxy groups will usually have from 1 to 6 carbon atoms attached through the oxygen bridge. “Lower alkoxy” refers to alkoxy groups having 1 to 4 carbons.

“Aryl” encompasses:
5- and 6-membered carbocyclic aromatic rings, for example, benzene;
bicyclic ring systems wherein at least one ring is carbocyclic and aromatic, for example, naphthalene, indane, and tetralin; and
tricyclic ring systems wherein at least one ring is carbocyclic and aromatic, for example, fluorene.
   For example, aryl includes 5- and 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

The term “halo” includes fluoro, chloro, bromo, and iodo, and the term “halogen” includes fluorine, chlorine, bromine, and iodine.

“Heteroaryl” encompasses:
5- to 7-membered aromatic, monocyclic rings containing one or more, for example, from 1 to 4, or In some embodiments, from 1 to 3, heteroatoms chosen from N, O, and S, with the remaining ring atoms being carbon; and
bicyclic heterocycloalkyl rings containing one or more, for example, from 1 to 4, or In some embodiments, from 1 to 3, heteroatoms chosen from N, O, and S, with the remaining ring atoms being carbon and wherein at least one heteroatom is present in an aromatic ring.
   For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridizinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above.

Substituted heteroaryl also includes ring systems substituted with one or more oxide (—O⁻) substituents, such as pyridinyl N-oxides.

By “heterocycloalkyl” is meant a single aliphatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperdyl, and 2,5-piperzinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently chosen from:

—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted aryl, and optionally substituted heteroaryl;

R^{b} is chosen from H, optionally substituted C₁-C₆ alkyl, optionally substituted aryl, and optionally substituted heteroaryl; and

R^{c} is chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted alkoxy” refers to alkoxy wherein the alkyl constituent is substituted (i.e., —O-(substituted alkyl)) wherein “substituted alkyl” is as described herein. “Substituted alkoxy” also includes glycosides (i.e., glycosyl groups) and derivatives of ascorbic acid. “Glycosides” refer to any of a number of sugar derivatives that contain a non-sugar group bonded to an oxygen or nitrogen atom of a sugar and that on hydrolysis yield that sugar. An example of a glycosyl group is glucosyl. “Derivatives of ascorbic acid” or “ascorbic acid derivatives” refer to any of a number of derviatives that contain a non-sugar group bonded to an oxygen or nitrogen atom of ascorbic acid and that on hydrolysis yield ascorbic acid (i.e., (R)-5-((S)-1,2-dihydroxyethyl)-3,4-dihydroxyfuran-2(5H)-one).

The term “alkoxycarbonyl” refers to a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality and wherein substituted alkyl is as described herein.

“Acyl” refers to the groups H—C(O)—, (alkyl)-C(O)—, (aryl)-C(O)—, (heteroaryl)-C(O)—, and (heterocycloalkyl)-C(O)—, wherein the group is attached to the parent structure through the carbonyl functionality, and wherein alkyl, aryl, heteroaryl, and heterocycloalkyl are optionally substituted as described herein. Examples include acetyl, benzoyl, propionyl, isobutyryl, t-butoxycarbonyl, benzyloxycarbonyl and the like. “Lower-acyl” refers to groups containing one to six carbons and “acyloxy” refers to the group O-acyl.

The term “amino” refers to the group —NH₂.

The term “substituted amino” refers to the group —NHR^{d} or —NR^{d}R^{e} wherein

R^{d} is chosen from hydroxy, optionally substituted alkoxy, optionally substituted alkyl, optionally substituted cycloalkyl, optionally substituted acyl, optionally substituted carbamimidoyl, aminocarbonyl, optionally substituted aryl, optionally substituted heteroaryl, optionally substituted heterocycloalkyl, optionally substituted alkoxycarbonyl, sulfinyl and sulfonyl, and

R^{e} is chosen from optionally substituted alkyl, optionally substituted cycloalkyl, optionally substituted aryl, optionally substituted heteroaryl, and optionally substituted heterocycloalkyl, and wherein

substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently chosen from:

—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NcN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{c}), where

R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{b} is independently chosen from hydrogen and optionally substituted C₁-C₄ alkyl; or R^{b} and R^{c}, and the nitrogen to which they are attached, form an optionally substituted heterocycloalkyl group; and where each optionally substituted group is unsubstituted or independently substituted with one or more, such as one, two, or three, substituents independently chosen from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄—NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl); and wherein

optionally substituted acyl, optionally substituted alkoxycarbonyl, sulfinyl and sulfonyl are as defined herein.

The term “sulfanyl” refers to the groups: —S-(optionally substituted alkyl), —S-(optionally substituted cycloalkyl), —S-(optionally substituted aryl), —S-(optionally substituted heteroaryl), and —S-(optionally substituted heterocycloalkyl).

The term “sulfinyl” refers to the groups: —S(O)—H, —S(O)-(optionally substituted alkyl), —S(O)-(optionally substituted cycloalkyl), —S(O)-(optionally substituted amino), —S(O)-(optionally substituted aryl), —S(O)-(optionally substituted heteroaryl), and —S(O)-(optionally substituted heterocycloalkyl), wherein substituted alkyl, substituted cycloalkyl, substituted amino, substituted aryl, substituted heteroaryl, and substituted heterocycloalkyl are as described herein.

The term “sulfonyl” refers to the groups: —S(O₂)—H, —S(O₂)-(optionally substituted alkyl), —S(O₂)-(optionally substituted cycloalkyl), —S(O₂)-(optionally substituted amino), —S(O₂)-(optionally substituted aryl), —S(O₂)-(optionally substituted heteroaryl), and —S(O₂)-(optionally substituted heterocycloalkyl), wherein substituted alkyl, substituted cycloalkyl, substituted amino, substituted aryl, substituted heteroaryl, and substituted heterocycloalkyl are as described herein.

Compounds described herein include, but are not limited to, their optical isomers, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

Chemical entities include, but are not limited to compounds described herein and all pharmaceutically acceptable forms thereof. Pharmaceutically acceptable forms of the compounds recited herein include pharmaceutically acceptable salts, prodrugs, and mixtures thereof. In some embodiments, the compounds described herein are in the form of pharmaceutically acceptable salts and prodrugs. Hence, the terms “chemical entity” and “chemical entities” also encompass pharmaceutically acceptable salts, prodrugs, and mixtures thereof.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, prodrugs also fall within the scope of chemical entities described herein. In some embodiments, the “prodrugs” described herein include any compound that becomes a compound of Formula I when administered to a patient, e.g., upon metabolic processing of the prodrug. Examples of prodrugs include derivatives of functional groups, such as a carboxylic acid group, in the compounds of Formula I. Exemplary prodrugs of a carboxylic acid group include, but are not limited to, carboxylic acid esters such as alkyl esters, hydroxyalkyl esters, arylalkyl esters, and aryloxyalkyl esters. Other exemplary prodrugs include lower alkyl esters such as ethyl ester, acyloxyalkyl esters such as pivaloyloxymethyl (POM), glycosides, and ascorbic acid derivatives.

Other exemplary prodrugs include amides of carboxylic acids. Exemplary amide prodrugs include metabolically labile amides that are formed, for example, with an amine and a carboxylic acid. Exemplary amines include NH₂, primary, and secondary amines such as NHR^{x}, and NR^{x}R^{y}, wherein R^{x} is hydrogen, (C₁-C₁₈)-alkyl, (C₃-C₇)-cycloalkyl, (C₃-C₇)-cycloalkyl-(C₁-C₄)-alkyl-, (C₆-C₁₄)-aryl which is unsubstituted or substituted by a residue (C₁-C₂)-alkyl, (C₁-C₂)-alkoxy, fluoro, or chloro; heteroaryl-, (C₆-C₁₄)-aryl-(C₁-C₄)-alkyl- where aryl is unsubstituted or substituted by a residue (C₁-C₂)-alkyl, (C₁-C₂)-alkoxy, fluoro, or chloro; or heteroaryl-(C₁-C₄)-alkyl- and in which R^{y} has the meanings indicated for R^{x} with the exception of hydrogen or wherein R^{x} and R^{y}, together with the nitrogen to which they are bound, form an optionally substituted 4- to 7-membered heterocycloalkyl ring which optionally includes one or two additional heteroatoms chosen from nitrogen, oxygen, and sulfur. A discussion of prodrugs is provided in T. Higuchi and V. Stella, Pro-drugs as Novel Delivery Systems, Vol. 14 of the A.C.S. Symposium Series, in Edward B. Roche, ed., Bioreversible Carriers in Drug Design, American Pharmaceutical Association and Pergamon Press, 1987, and in Design of Prodrugs, ed. H. Bundgaard, Elsevier, 1985.

A “solvate” is formed by the interaction of a solvent and a compound. The term “compound” is intended to include solvates of compounds. Similarly, “salts” includes solvates of salts. Suitable solvates are pharmaceutically acceptable solvates, such as hydrates, including monohydrates and hemi-hydrates.

A “chelate” is formed by the coordination of a compound to a metal ion at two (or more) points. The term “compound” is intended to include chelates of compounds. Similarly, “salts” includes chelates of salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound’.

The term “hydrogen bond” refers to a form of association between an electronegative atom (also known as a hydrogen bond acceptor) and a hydrogen atom attached to a second, relatively electronegative atom (also known as a hydrogen bond donor). Suitable hydrogen bond donor and acceptors are well understood in medicinal chemistry (G. C. Pimentel and A. L. McClellan, The Hydrogen Bond, Freeman, San Francisco, 1960; R. Taylor and O. Kennard, “Hydrogen Bond Geometry in Organic Crystals”, Accounts of Chemical Research, 17, pp. 320-326 (1984)).

“Hydrogen bond acceptor” refers to a group comprising an oxygen or nitrogen, such as an oxygen or nitrogen that is sp²-hybridized, an ether oxygen, or the oxygen of a sulfoxide or N-oxide.

The term “hydrogen bond donor” refers to an oxygen, nitrogen, or heteroaromatic carbon that bears a hydrogen. group containing a ring nitrogen or a heteroaryl group containing a ring nitrogen.

As used herein the terms “group”, “radical” or “fragment” are synonymous and are intended to indicate functional groups or fragments of molecules attachable to a bond or other fragments of molecules.

The term “active agent” is used to indicate a chemical entity which has biological activity. In some embodiments, an “active agent” is a compound having pharmaceutical utility. For example an active agent may be an anti-neurodegenerative therapeutic.

The term “therapeutically effective amount” of a chemical entity described herein means an amount effective, when administered to a human or non-human patient, to provide a therapeutic benefit such as amelioration of symptoms, slowing of disease progression, or prevention of disease e.g., a therapeutically effective amount may be an amount sufficient to decrease the symptoms of a disease responsive to inhibition of KMO activity. In some embodiments, a therapeutically effective amount is an amount sufficient to treat the symptoms of neurodegenerative pathway or disease, such as Huntington's disease, Alzheimer's disease, Parkinson's disease, olivoponto cerebellar atrophy, non-Alzheimer's dementia, multi-infarctual dementia, cerebral amyotrophic lateral sclerosis, cerebral ischemia, cerebral hypoxia, spinal or head trauma, or epilepsy. In some embodiments a therapeutically effective amount is an amount sufficient to reduce the signs or side effects of a neurodegenerative disease. In some embodiments, a therapeutically effective amount of a chemical entity is an amount sufficient to prevent a significant increase or significantly reduce the level of neuronal cell death. In some embodiments a therapeutically effective amount is an amount sufficient to reduce the signs or side effects of a neurodegenerative disease. In some embodiments, a therapeutically effective amount of a chemical entity is an amount sufficient to prevent a significant increase or significantly reduce the level of QUIN associated with neuronal cell death. In some embodiments, a therapeutically effective amount of a chemical entity is an amount sufficient to effect an increase in the level of KYNA associated with neuronal cell health. In some embodiments, a therapeutically effective amount of a chemical entity is an amount sufficient to increase the anticonvulsant and neuroprotective properties associated with lowered levels of QUIN and increased levels of KYNA.

In methods described herein for treating a neurodegenerative disorder, a therapeutically effective amount may also be an amount sufficient, when administered to a patient, to detectably slow the progression of the neurodegenative disease, or prevent the patient to whom the chemical entity is given from presenting symptoms of the neurodegenative disease. In some methods described herein for treating a neurodegenative disease, a therapeutically effective amount may also be an amount sufficient to produce a detectable decrease in the level of neuronal cell death. For example, in some embodiments a therapeutically effective amount is an amount of a chemical entity described herein sufficient to significantly decrease the level of neuronal death by effecting a detectable decrease in the amount of QUIN, and an increase in the amount of KYNA.

The term “inhibition” indicates a significant decrease in the baseline activity of a biological activity or process. “Inhibition of KMO activity” refers to a decrease in KMO activity as a direct or indirect response to the presence of at least one chemical entity described herein, relative to the activity of KMO in the absence of at least one chemical entity. The decrease in activity may be due to the direct interaction of the compound with KMO, or due to the interaction of the chemical entity(ies) described herein with one or more other factors that in turn affect KMO activity. For example, the presence of the chemical entity(ies) may decrease KMO activity by directly binding to the KMO, by causing (directly or indirectly) another factor to decrease KMO activity, or by (directly or indirectly) decreasing the amount of KMO present in the cell or organism.

“Inhibition of KMO activity” refers to a decrease in KMO activity as a direct or indirect response to the presence of at least one chemical entity described herein, relative to the activity of KMO in the absence of the at least one chemical entity. The decrease in activity may be due to the direct interaction of the compound with KMO or with one or more other factors that in turn affect KMO activity.

Inhibition of KMO activity also refers to an observable inhibition of 3-HK and QUIN production in a standard assay such as the assay described below. The inhibition of KMO activity also refers to an observable increase in the production of KYNA. In some embodiments, the chemical entity described herein has an IC₅₀ value less than or equal to 1 micromolar. In some embodiments, the chemical entity has an IC₅₀ value less than or equal to less than 100 micromolar. In some embodiments, the chemical entity has an IC₅₀ value less than or equal to 10 nanomolar.

“KMO activity” also includes activation, redistribution, reorganization, or capping of one or more various KMO membrane receptors, or receptor sites can undergo redistribution and capping that can initiate signal transduction. KMO activity also includes the synthesis or production of QUIN and 3-HK.

A “disease responsive to inhibition of KMO activity” is a disease in which inhibiting KMO provides a therapeutic benefit such as an amelioration of symptoms, decrease in disease progression, prevention or delay of disease onset, or inhibition of aberrant activity and/or death of certain cell-types (neuronal cells).

“Treatment” or “treating” means any treatment of a disease in a patient, including:
a) preventing the disease, that is, causing the clinical symptoms of the disease not to develop;
b) inhibiting the disease;
c) slowing or arresting the development of clinical symptoms; and/or
d) relieving the disease, that is, causing the regression of clinical symptoms.

“Subject” or “patient’ refers to an animal, such as a mammal, that has been or will be the object of treatment, observation or experiment. The methods described herein may be useful in both human therapy and veterinary applications. In some embodiments, the subject is a mammal; and in some embodiments the subject is human.

Provided is at least one chemical entity chosen from compounds of formula I

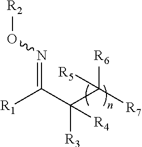

and pharmaceutically acceptable salts and prodrugs thereof wherein:
R₁ is chosen from aryl and heteroaryl, each of which is substituted with one, two, or three groups chosen from halo, lower alkyl, alkoxy, and hydroxy;
R₂ is chosen from hydrogen and optionally substituted lower alkyl;
R₃ and R₄ are independently chosen from hydrogen, halo, hydroxy, lower alkyl, and lower alkoxy;
for each occurrence, R₅ and R₆ are independently chosen from hydrogen and lower alkyl;
or R₃ and R₅, taken together with the atoms to which they are attached, form an optionally substituted cycloalkyl ring,
n is one or two;
R₇ is chosen from —C(O)OR₈, —C(O)R₈, optionally substituted amino, —C(═N—OR₁₁)R₈, —C(O)NR₉R₁₀, cyano, and optionally substituted heteroaryl;
R₈ is chosen from hydrogen, optionally substituted lower alkyl, optionally substituted aryl, optionally substituted heteroaryl, optionally substituted cycloalkyl, optionally substituted heterocycloalkyl, and glycosyl; and
R₉ and R₁₀ are independently chosen from hydrogen, optionally substituted lower alkyl, optionally substituted lower alkoxy, optionally substituted cycloalkyl, optionally substituted aryl, optionally substituted heterocycloalkyl, and optionally substituted heteroaryl;
or R₉ and R₁₀, together with the nitrogen to which they are bound, form an optionally substituted heterocycloalkyl or optionally substituted heteroaryl ring;
R₁₁ is chosen from hydrogen and optionally substituted lower alkyl,
provided that if R₁ is 4-bromophenyl, 4-fluorophenyl, 4-methoxyphenyl, 4-methylphenyl, 3-methoxyphenyl, 3-chloro-4-n-propoxyphenyl, 3-chloro-4-ethoxyphenyl, 3-chloro-4-n-butoxyphenyl, 2,3,4-trimethylphenyl, 3,4,5-trimethylphenyl, 3-chloro-4-methoxyphenyl, 2,4-dimethoxyphenyl, or 2-hydroxy-5-methylphenyl;
   n is 1;
   R₂, R₃, R₄, R₅, and R₆ are hydrogen; and
   R₇ is —C(O)OR₈, then R₈ is not methyl, ethyl, or hydrogen;
provided that if R₁ is 4-fluorophenyl;
   n is 1;
   R₂, R₃, R₄ and R₅ are hydrogen;
   R₆ is methyl; and
   R₇ is —C(O)OR₈, then R₈ is not ethyl;
provided that if R₁ is 4-chlorophenyl;
   n is 1;
   R₂, R₃, R₄ and R₅ are hydrogen and R₆ is methyl, ethyl, or butyl; or
   R₂, R₃, and R₄ and R₅ and R₆ are ethyl; and
   R₇ is —C(O)OR₈, then R₈ is not ethyl;
provided that if R₁ is 5-bromo-2-methoxyphenyl;
   n is 2;
   R₂, R₃, R₄, R₅, and R₆ are hydrogen; and
   R₇ is —C(O)OR₈, then R₈ is not methyl or ethyl;
provided that if R₁ is 2-hydroxyphenyl, 4-hydroxyphenyl, 2,4-dihydroxy-6-methylphenyl, 4-ethoxyphenyl, or 4-methoxyphenyl;
   n is 2;
   R₂, R₃, R₄, R₅, and R₆ are each hydrogen; and
   R₇ is —C(O)OR₈, then R₈ is not hydrogen;
provided that if R₁ is 3-chloro-4-methoxyphenyl or 2,5-dimethylphenyl;
   n is 1;
   R₂, R₃, R₄, and R₅ are hydrogen;
   R₆ is ethyl or propyl; and
   R₇ is —C(O)OR₈, then R₈ is not hydrogen;
provided that if R₁ is 4-methylphenyl;
   n is 1;
   R₂, R₃, R₄ and R₅ are hydrogen;
   R₅ is methyl; and
   R₇ is —C(O)OR₈, then R₈ is not hydrogen; and
provided that if R₁ is 4-methoxyphenyl;
   n is 1;
   R₂, R₄, R₅ and R₆ are hydrogen;
   R₃ is methyl; and
   R₇ is —C(O)OR₈, then R₈ is not hydrogen.

Provided is at least one chemical entity chosen from compounds of formula I

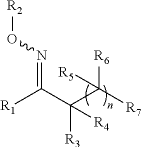

and pharmaceutically acceptable salts and prodrugs thereof wherein:
R₁ is chosen from aryl and heteroaryl, each of which is substituted with one, two, or three groups chosen from halo, lower alkyl, alkoxy, and hydroxy;
R₂ is chosen from hydrogen and optionally substituted lower alkyl;
R₃ and R₄ are independently chosen from hydrogen, halo, hydroxy, lower alkyl, and lower alkoxy;
for each occurrence, R₅ and R₆ are independently chosen from hydrogen and lower alkyl;
or R₃ and R₅, taken together with the atoms to which they are attached, form an optionally substituted cycloalkyl ring,
n is one or two;
R₇ is chosen from —C(O)OR₈, —C(O)NR₉R₁₀, and optionally substituted heteroaryl;
R₈ is chosen from hydrogen, optionally substituted lower alkyl, optionally substituted aryl, optionally substituted heteroaryl, optionally substituted cycloalkyl, optionally substituted heterocycloalkyl, and glycosyl; and
R₉ and R₁₀ are independently chosen from hydrogen, optionally substituted lower alkyl, optionally substituted cycloalkyl, optionally substituted aryl, optionally substituted heterocycloalkyl, and optionally substituted heteroaryl;
or R₉ and R₁₀, together with the nitrogen to which they are bound, form an optionally substituted heterocycloalkyl or optionally substituted heteroaryl ring,
provided that if R₁ is 4-bromophenyl, 4-fluorophenyl, 4-methoxyphenyl, 4-methylphenyl, 3-methoxyphenyl, 3-chloro-4-n-propoxyphenyl, 3-chloro-4-ethoxyphenyl, 3-chloro-4-n-butoxyphenyl, 2,3,4-trimethylphenyl, 3,4,5-trimethylphenyl, 3-chloro-4-methoxyphenyl, 2,4-dimethoxyphenyl, or 2-hydroxy-5-methylphenyl;
   n is 1;
   R₂, R₃, R₄, R₅, and R₆ are hydrogen; and
   R₇ is —C(O)OR₈, then R₈ is not methyl, ethyl, or hydrogen;
provided that if R₁ is 4-fluorophenyl;
   n is 1;
   R₂, R₃, R₄ and R₅ are hydrogen;
   R₆ is methyl; and
   R₇ is —C(O)OR₈, then R₈ is not ethyl;
provided that if R₁ is 4-chlorophenyl;
   n is 1;
   R₂, R₃, R₄ and R₅ are hydrogen and R₆ is methyl, ethyl, or butyl; or
   R₂, R₃, and R₄ and R₅ and R₆ are ethyl; and
   R₇ is —C(O)OR₈, then R₈ is not ethyl;
provided that if R₁ is 5-bromo-2-methoxyphenyl;
   n is 2;
   R₂, R₃, R₄, R₅, and R₆ are hydrogen; and
   R₇ is —C(O)OR₈, then R₈ is not methyl or ethyl;
provided that if R₁ is 2-hydroxyphenyl, 4-hydroxyphenyl, 2,4-dihydroxy-6-methylphenyl, 4-ethoxyphenyl, or 4-methoxyphenyl;
   n is 2;
   R₂, R₃, R₄, R₅, and R₆ are each hydrogen; and
   R₇ is —C(O)OR₈, then R₈ is not hydrogen;
provided that if R₁ is 3-chloro-4-methoxyphenyl or 2,5-dimethylphenyl;
   n is 1;
   R₂, R₃, R₄, and R₅ are hydrogen;
   R₆ is ethyl or propyl; and
   R₇ is —C(O)OR₈, then R₈ is not hydrogen;
provided that if R₁ is 4-methylphenyl;
   n is 1;
   R₂, R₃, R₄ and R₅ are hydrogen;
   R₆ is methyl; and
   R₇ is —C(O)OR₈, then R₈ is not hydrogen; and
provided that if R₁ is 4-methoxyphenyl;
   n is 1;
   R₂, R₄, R₅ and R₆ are hydrogen;
   R₃ is methyl; and
   R₇ is —C(O)OR₈, then R₈ is not hydrogen.

In some embodiments, the compound of Formula I is the syn isomer. In some embodiments, the compound of Formula I is the anti isomer.

In some embodiments, R₁ is chosen from optionally substituted phenyl and optionally substituted heteroaryl. In some embodiments, R₁ is chosen from pyridinyl and phenyl substituted with one, two or three halo groups. In some embodiments, R₁ is phenyl substituted with one or two halo groups. In some embodiments, R₁ is 3,4-dihalophenyl. In some embodiments, R₁ is 3,4-dichlorophenyl.

In some embodiments, R₂ is chosen from hydrogen, lower alkyl and lower alkyl substituted with one to three substituents independently chosen from optionally substituted amino, optionally substituted aryl, and optionally substituted cycloalkyl. In some embodiments, R₂ is chosen from hydrogen, lower alkyl, lower alkyl substituted with cycloalkyl, lower alkyl substituted with optionally substituted phenyl, and —NR₁₂R₁₂ wherein, for each occurrence, R₁₂ is chosen from hydrogen, alkyl, aralkyl, cycloalkyl, heteroaralkyl, heterocycloalkyl, aryl, heteroaryl, and acyl. In some embodiments, R₂ is chosen from hydrogen and lower alkyl. In some embodiments, R₂ is methyl.

In some embodiments, at least one of R₃ and R₄ is hydrogen. In some embodiments, R₃ and R₄ are hydrogen.

In some embodiments, at least one of R₃ and R₄ is fluoro. In some embodiments, at least one of R₃ and R₄ is methyl. In some embodiments, at least one of R₃ and R₄ is methoxy.

In some embodiments, for each occurrence, at least one of R₅ and R₆ is hydrogen. In some embodiments, for each occurrence, R₅ and R₆ are hydrogen.

In some embodiments, n is one. In some embodiments, n is two.

In some embodiments, R₃ and R₅, taken together with the atoms to which they are attached, form an optionally substituted cyclopropyl ring.

In some embodiments, R₇ is —C(O)OR₈. In some embodiments, R₇ is —C(O)NR₉R₁₀.

In some embodiments, R₈ is chosen from hydrogen and lower alkyl. In some embodiments, R₈ is chosen from hydrogen and methyl. In some embodiments, R₈ is glycosyl. In some embodiments, R₈ is an ascorbic acid derivative.

In some embodiments, R₉ and R₁₀ are independently chosen from hydrogen, optionally substituted lower alkyl, optionally substituted cycloalkyl, optionally substituted aryl, optionally substituted heterocycloalkyl, and optionally substituted heteroaryl.

In some embodiments, R₉ is hydrogen or lower alkyl. In some embodiments, R₉ is hydrogen.

In some embodiments, R₁₀ is chosen from optionally substituted lower alkyl, optionally substituted cycloalkyl, optionally substituted aryl, optionally substituted heterocycloalkyl, and optionally substituted heteroaryl. In some embodiments, R₁₀ is chosen from optionally substituted lower alkyl, optionally substituted cycloalkyl, optionally substituted phenyl, optionally substituted heterocycloalkyl, and optionally substituted pyridinyl. In some embodiments, R₁₀ is chosen from optionally substituted thiadiazolyl, optionally substituted oxadiazolyl, optionally substituted tetrazolyl, optionally substituted pyrrolidinyl, optionally substituted pyrimidinyl, optionally substituted pyridinyl, optionally substituted pyridazinyl, optionally substituted pyrazinyl, and optionally substituted oxazolyl. In some embodiments, R₁₀ is chosen from optionally substituted thiadiazolyl, optionally substituted diazolyl, optionally substituted tetrazolyl, and optionally substituted oxazolyl.

In some embodiments, R₉ and R₁₀, together with the nitrogen to which they are bound, form an optionally substituted heterocycloalkyl ring. In some embodiments, R₉ and R₁₀, together with the nitrogen to which they are bound, form an optionally substituted pyrrolidinyl, optionally substituted morpholinyl, optionally substituted piperazinyl, or optionally substituted piperidinyl ring. In some embodiments, R₉ and R₁₀, together with the nitrogen to which they are bound, form an optionally substituted morpholinyl, optionally substituted piperazinyl, or optionally substituted piperidinyl ring. In some embodiments, R₇ is chosen from optionally substituted imidazopyridinyl, optionally substituted indazoyl, optionally substituted oxadiazolyl, optionally substituted triazolyl, optionally substituted isoxazolyl, and optionally substituted tetrazolyl.

In some embodiments, R₇ is —C(═N—OR₁₁)R₈, wherein R₁₁ is lower alkyl and R₈ is lower alkyl.

Also provided is at least one chemical entity chosen from
4-(3,4-Dichloro-phenyl)-4-methoxyimino-butyric acid;
4-(3,4-Dichloro-phenyl)-4-ethoxyimino-butyric acid;
4-Benzyloxyimino-4-(3,4-dichloro-phenyl)-butyric acid;
4-Cyclopropylmethoxyimino-4-(3,4-dichloro-phenyl)-butyric acid;
4-(3,5-Dichloro-phenyl)-4-methoxyimino-butyric acid;
4-(3,4-Dichloro-phenyl)-4-methoxyimino-2-methyl-butyric acid;
4-(3,4-Dichloro-phenyl)-4-methoxyimino-3-methyl-butyric acid;
4-Methoxyimino-4-pyridin-2-yl-butyric acid;
4-Methoxyimino-4-pyridin-3-yl-butyric acid;
4-(3,4-Dichloro-phenyl)-4-methoxyimino-butyric acid methyl ester;
4-(3,4-Dichloro-phenyl)-4-ethoxyimino-butyric acid methyl ester;
4-Benzyloxyimino-4-(3,4-dichloro-phenyl)-butyric acid methyl ester;
4-(3,4-Dichloro-phenyl)-4-hydroxyimino-butyric acid methyl ester;
5-(3,4-Dichloro-phenyl)-5-methoxyimino-pentanoic acid methyl ester;
5-(3,4-Dichloro-phenyl)-5-methoxyimino-pentanoic acid;
1-(3,4-Dichloro-phenyl)-5-methoxy-pentan-1-one O-methyl-oxime;
4-(3,4-Dichloro-phenyl)-4-methoxyimino-N-phenyl-butyramide;
4-(3,4-Dichloro-phenyl)-4-methoxyimino-N-pyridin-3-yl-butyramide;
4-(3,4-Dichloro-phenyl)-4-methoxyimino-N-(2-morpholin-4-yl-ethyl)-butyramide;
4-(3,4-Dichloro-phenyl)-4-methoxyimino-N-[1,3,4]thiadiazol-2-yl-butyramide;
4-(3,4-Dichloro-phenyl)-4-methoxyimino-N-pyridin-4-yl-butyramide;
4-(3,4-Dichloro-phenyl)-4-methoxyimino-N-pyridin-2-yl-butyramide;
4-(3,4-Dichloro-phenyl)-N-isoxazol-3-yl-4-methoxyimino-butyramide;
4-(3,4-Dichloro-phenyl)-4-methoxyimino-N-(1-methyl-1H-pyrazol-4-yl)-butyramide;
1-(3,4-Dichloro-phenyl)-4-morpholin-4-yl-butane-1,4-dione 1-(O-methyl-oxime);
4-(3,4-Dichloro-phenyl)-N-ethyl-4-methoxyimino-butyramide;
4-(3,4-Dichloro-phenyl)-N-isopropyl-4-methoxyimino-butyramide;
N-Cyclopropyl-4-(3,4-dichloro-phenyl)-4-methoxyimino-butyramide;
4-(3,4-Dichloro-phenyl)-4-methoxyimino-N-methyl-butyramide;
4-[4-(3,4-Dichloro-phenyl)-4-methoxyimino-butyrylamino]-piperidine-1-carboxylic acid tert-butyl ester;
4-[4-(3,4-Dichloro-phenyl)-4-methoxyimino-butyrylamino]-piperidinium trifluoroacetate;
{2-[4-(3,4-Dichloro-phenyl)-4-methoxyimino-butyrylamino]-ethyl}-dimethyl-ammonium trifluoroacetate;
4-(3,4-Dichloro-phenyl)-4-methoxyimino-N,N-dimethyl-butyramide;
4-(3,4-Dichloro-phenyl)-4-methoxyimino-2-methyl-N-phenyl-butyramide;
4-(3,4-Dichloro-phenyl)-4-methoxyimino-2-methyl-N-pyridin-3-yl-butyramide;
4-(3,4-Dichloro-phenyl)-4-methoxyimino-butyric acid isopropyl ester;
4-(3,4-Dichloro-phenyl)-4-methoxyimino-butyric acid cyclopentyl ester;
4-(3,4-Dichloro-phenyl)-4-methoxyimino-butyric acid cyclobutyl ester;
4-(3,4-Dichloro-phenyl)-4-methoxyimino-butyric acid pyridin-3-yl ester;
4-(3,4-Dichloro-phenyl)-4-methoxyimino-butyric acid tetrahydro-pyran-4-yl ester;
1-(3,4-Dichloro-phenyl)-3-(3-methyl-[1,2,4]oxadiazol-5-yl)-propan-1-one O-methyl-oxime;
1-(3,4-Dichloro-phenyl)-3-(2H-tetrazol-5-yl)-propan-1-one O-methyl-oxime;
1-(3,4-Dichloro-phenyl)-3-imidazol-1-yl-propan-1-one O-methyl-oxime;
1-(3,4-Dichloro-phenyl)-4-imidazol-1-yl-butan-1-one O-methyl-oxime;
1-(3,4-Dichloro-phenyl)-3-(5-methyl-isoxazol-3-yl)-propan-1-one O-methyl-oxime;
(1S,2S)-trans-2-[(3,4-Dichloro-phenyl)-methoxyimino-methyl]-cyclopropanecarboxylic acid methyl ester;
(1S,2S)-trans-2-[(3,4-Dichloro-phenyl)-methoxyimino-methyl]-cyclopropanecarboxylic acid; and
4-(3,4-Dichloro-phenyl)-4-methoxyimino-butyric acid 3,4,5,6-tetrahydroxy-tetrahydro-pyran-2-ylmethyl ester,
or a pharmaceutically acceptable salt or prodrug thereof.

Also provided is at least one chemical entity chosen from
4-[2-tert-Butoxycarbonylamino-ethoxyimino]-4-(3,4-dichloro-phenyl)-butyric acid;
4-[2-Amino-ethoxyimino]-4-(3,4-dichloro-phenyl)-butyric acid;
4-(3,4-Dichloro-phenyl)-4-(2-dimethylamino-ethoxyimino)-butyric acid;
4-(4,5-Dichloro-2-hydroxy-phenyl)-4-hydroxyimino-butyric acid;
4-(3,4-Dichloro-phenyl)-4-methoxyimino-butyramide;
4-(3,4-Dichloro-phenyl)-4-methoxyimino-butyronitrile;
4-(3-Chloro-phenyl)-4-methoxyimino-butyric acid;
4-(3,4-Dichloro-phenyl)-4-methoxyimino-N-pyrazin-2-yl-butyramide;
4-(3,4-Dichloro-phenyl)-4-methoxyimino-N-pyridin-3-ylmethyl-butyramide;
4-(3,4-Dichloro-phenyl)-4-methoxyimino-N-(4-methyl-pyridin-3-yl)-butyramide;
1-(3,4-DiChloro-phenyl)-3-(2-methyl-2H-tetrazol-5-yl)-propan-1-one O-methyl-oxime;
1-(3,4-Dichloro-phenyl)-3-(2-methyl-2H-tetrazol-5-yl)-propan-1-one O-methyl-oxime;
1-(3,4-Dichloro-phenyl)-3-(5-methyl-[1,2,4]oxadiazol-3-yl)-propan-1-one O-methyl-oxime;
1-(3,4-Dichloro-phenyl)-3-[1,2,4]triazol-1-yl-propan-1-one O-methyl-oxime;
4-(3,4-Dichloro-phenyl)-4-methoxyimino-N-pyrimidin-5-yl-butyramide;
4-(3,4-Dichloro-phenyl)-4-methoxyimino-N-(5-methyl-pyridin-3-yl)-butyramide;
4-(3,4-Dichloro-phenyl)-N-(2,6-dimethyl-pyridin-3-yl)-4-methoxyimino-butyramide;
4-(3,4-Dichloro-phenyl)-4-methoxyimino-N-(2-methyl-pyrimidin-5-yl)-butyramide;
1-(3,4-Dichloro-phenyl)-3-(3-hydroxy-isoxazol-5-yl)-propan-1-one O-methyl-oxime;
4-(3,4-Dichloro-phenyl)-N-(5-fluoro-pyridin-3-yl)-4-methoxyimino-butyramide;
4-(3,4-Dichloro-phenyl)-4-methoxyimino-N-pyridazin-3-yl-butyramide;
4-(3,4-Dichloro-phenyl)-N-(3,5-dimethyl-pyrazin-2-yl)-4-methoxyimino-butyramide;
4-(3,4-Dichloro-phenyl)-4-methoxyimino-N-(6-trifluoromethyl-pyridin-3-yl)-butyramide;
1-(3,4-Dichloro-phenyl)-4-piperidin-1-yl-butane-1,4-dione 1-(O-methyl-oxime);
1-(3,4-Dichloro-phenyl)-4-(4-methyl-piperidin-1-yl)-butane-1,4-dione 1-(O-methyl-oxime);
4-(3,4-Dichloro-phenyl)-4-methoxyimino-N-(6-methyl-pyridazin-3-yl)-butyramide;
4-(3,4-Dichloro-phenyl)-4-methoxyimino-N-(6-methyl-pyridin-3-yl)-butyramide;
1-(3,4-Dichloro-phenyl)-4-(3-hydroxy-pyrrolidin-1-yl)-butane-1,4-dione 1-(O-methyl-oxime);
4-(3,4-Dichloro-phenyl)-4-methoxyimino-N-pyrimidin-2-yl-butyramide;
Pyrimidine-5-carboxylic acid {3-(3,4-dichloro-phenyl)-3-methoxyimino-propyl}-amide;
4-(3,4-Dichloro-phenyl)-N-(2-hydroxy-ethyl)-4-methoxyimino-butyramide;
5-(3,4-Dichloro-phenyl)-5-methoxyimino-pentanoic acid amide;
1-(3,4-Dichloro-phenyl)-4-(4-hydroxy-piperidin-1-yl)-butane-1,4-dione1-(O-methyl-oxime);
3-{4-(3,4-Dichloro-phenyl)-4-methoxyimino-butyrylamino}-pyrrolidine-1-carboxylic acid tert-butyl ester;
4-(3,4-Dichloro-phenyl)-N-(2-hydroxy-propyl)-4-methoxyimino-butyramide;
4-(3,4-Dichloro-phenyl)-N-(2,4-dimethyl-pyridin-3-yl)-4-methoxyimino-butyramide;
5-(3,4-Dichloro-phenyl)-5-methoxyimino-pentanenitrile;
4-(3,4-Dichloro-phenyl)-N-(2,3-dihydroxy-propyl)-4-methoxyimino-butyramide;
4-(3,4-Dichloro-phenyl)-4-methoxyimino-N-pyrrolidin-3-yl-butyramide;
4-(3,4-Dichloro-phenyl)-N-methoxy-4-methoxyimino-N-methyl-butyramide;
1-(3,4-Dichloro-phenyl)-pentane-1,4-dione 1-(O-methyl-oxime);
4-(3,4-Dichloro-phenyl)-N-(6-fluoro-pyridin-3-yl)-4-methoxyimino-butyramide;
N-{3-(3,4-Dichloro-phenyl)-3-methoxyimino-propyl}-methanesulfonamide;
1-(3,4-Dichloro-phenyl)-pentane-1,4-dione bis-(O-methyl-oxime);
4-(3,4-Dichloro-phenyl)-4-methoxyimino-N-(2,4,6-trimethyl-pyridin-3-yl)-butyramide;
Pyridine-3-sulfonic acid {3-(3,4-dichloro-phenyl)-3-methoxyimino-propyl}-amide;
4-(3,4-Dichloro-phenyl)-4-methoxyimino-butyric acid (S)-2-((R)-3,4-dihydroxy-5-oxo-2,5-dihydro-furan-2-yl)-2-hydroxy-ethyl ester; and
1-(3,4-Dichloro-phenyl)-3-(1H-imidazo[4,5-c]pyridin-2-yl)-propan-1-one O-methyl-oxime ester,
or a pharmaceutically acceptable salt or prodrug thereof.

Methods for obtaining the chemical entities described herein will be apparent to those of ordinary skill in the art, suitable procedures being described, for example, in the reaction schemes and examples below, and in the references cited herein.

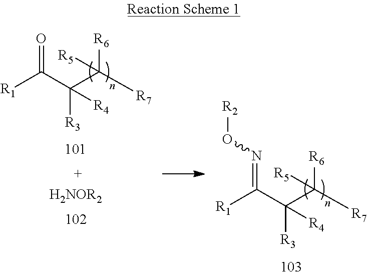

Referring to Reaction Scheme 1, to a solution of a compound of Formula 101 in a polar solvent such as EtOH is added an excess of a compound of Formula 102 (such as about 5.0 equivalents) and a base such as pyridine (such as about 3.3 equivalents). The mixture is stirred at about 95° C. and may be monitored until completion. The product, a compound of Formula 103, is isolated and optionally purified.

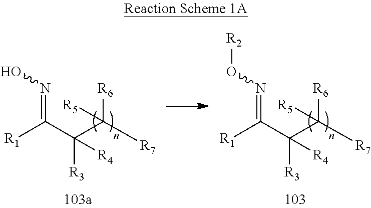

Referring to Reaction Scheme 1A, to a solution of a compound of Formula 103a is added a base, such as sodium hydride (such as about 1 eq. to about 2.1 eq.). The mixture is stirred for about 30 min. A compound of the formula R₂—X′, where X′ is a leaving group, for example bromine or chlorine, and NaI (such as about 0.05 eq.) is added. The mixture is stirred ambient temperature and may be monitored until completion. The product, a compound of Formula 103, is isolated and optionally purified.

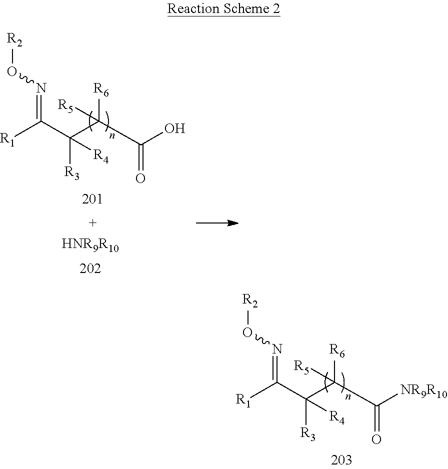

Referring to Reaction Scheme 2, to a solution of a carboxylic acid of Formula 201 in a solvent such as DMF is added EDC.HCl (such as about 1 equivalent) and a coupling agent (such as HOBt, TBTU, or DCC, such as about 1 equivalent). The reaction mixture is stirred at rt for about 30 min. An amine of Formula 202 (such as about 1.1 equivalents) is added to the reaction mixture, which may be monitored until completion. In some embodiments the mixture is heated between about 30° C. and 50° C. In some embodiments the mixture is heated at about 120° C. for about 30 min in a microwave. The resulting product, a compound of Formula 203, is isolated and optionally purified.

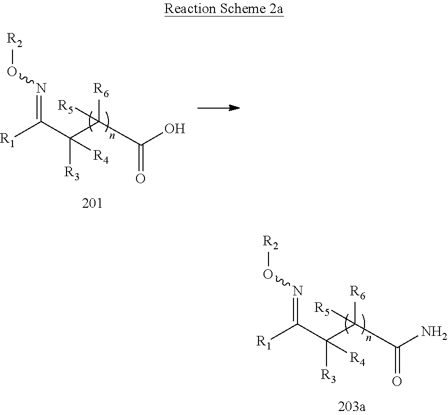

Referring to Reaction Scheme 2a, to a solution of a carboxylic acid of Formula 201 in a solvent such as THF is added 1,1′-carbonyldiimidazole (such as about 1.1 equivalent) and the mixture is stirred for about 30 min. A solution of ammonia (such as about 0.5M) in 1,4 dioxane (such as about 18 equivalents) is added to the reaction mixture, which may be monitored until completion. The resulting product, a compound of Formula 203a, is isolated and optionally purified.

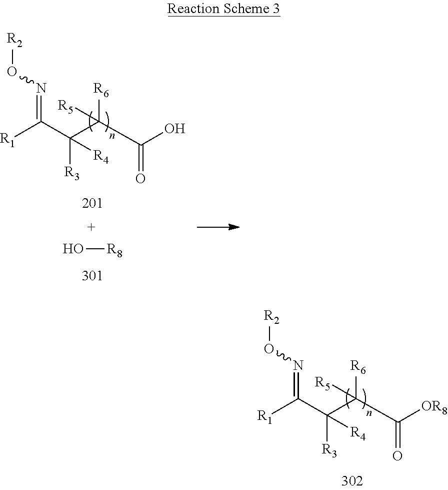

Referring to Reaction Scheme 3, a compound of Formula 201 and CDI (such as about 1.0 equivalent) are taken up in a polar solvent such as THF. The mixture is stirred at rt for about 30 min. An alcohol of Formula 301 is then added and the reaction is stirred at rt for about 16 hrs. The resulting product, a compound of Formula 302, is isolated and optionally purified.

Alternatively, referring to Reaction Scheme 3, to a solution of a compound of Formula 201 in sulfuric acid is added a compound of Formula 301 and the reaction is stirred at about 60° C. for about 1 hr. The resulting product, a compound of Formula 302, is isolated and optionally purified.

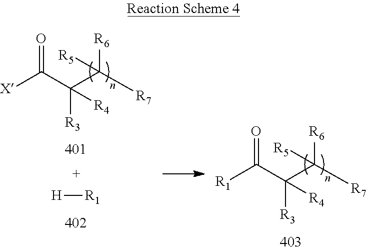

Referring to Reaction Scheme 4, to a stirred suspension of a compound of Formula 401 and a compound of Formula 402 (such as about 6 equivalents), where X′ is a leaving group such as chloride and R₁ represents an optionally-substituted aryl group such a halophenyl, is added a catalyst such as AlCl₃ in a solvent such as dichloroethane. The mixture is stirred at about 60° C. for about 2.5 hrs. The resulting product, a compound of Formula 403, is isolated and optionally purified.

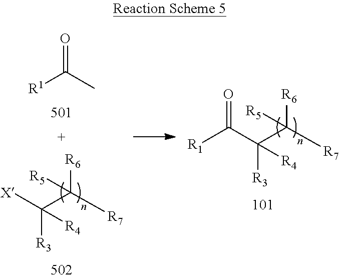

Referring to Reaction Scheme 5, a stirred solution of a compound of Formula 501 in a polar solvent such as THF is cooled to a temperature of about −55° C. To this solution is added a base such as LiHMDS (such as about 1.1 equivalents of a 1M solution), before cooling the solution to about −78° C. A compound of Formula 502 is then added, where X′ is a leaving group such as bromide. The reaction mixture is allowed to warm to rt and is stirred for about 96 hrs. The resulting product, a compound of Formula 101, is isolated and optionally purified.

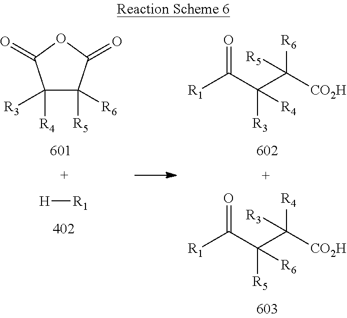

Referring to Reaction Scheme 6, to a stirred suspension of a compound of Formula 601 and a compound of Formula 402 (such as about 6 equivalents), where R₁ represents an optionally-substituted aryl group such as halophenyl, is added a catalyst such as AlCl₃. If the optionally-substituted aryl compound of Formula 402 is not a liquid, then a solvent such as dichloroethane may be added to form the suspension. The mixture is stirred at about 60° C. for about 2.5 hrs. The resulting products, compounds of Formulae 602 and 603, are isolated and optionally purified.

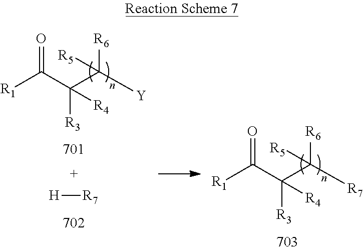

Referring to Reaction Scheme 7, to a stirred solution of a compound of Formula 702, where R₇ is a heteroaryl, is added a base, such as NaH (such as about 1.1 eq). The mixture is stirred for about 20 min. A compound of Formula 701, where Y is a leaving group such as chloride or bromide and R₁ represents an optionally-substituted aryl group such as a halophenyl is added and the mixture is stirred. The resulting product, a compound of Formula 703, is isolated and optionally purified.

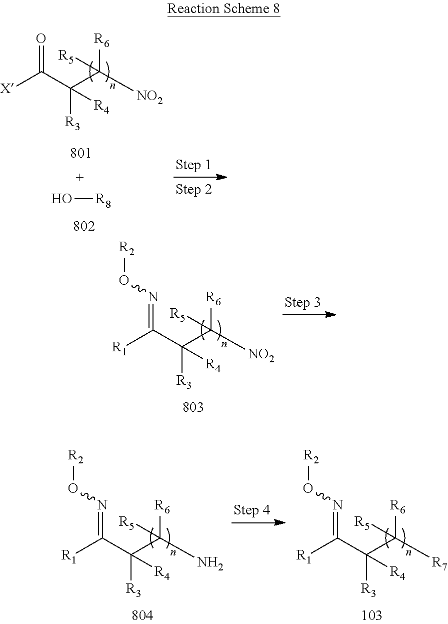

Referring to Reaction Scheme 8, Step 1, to a stirred suspension of a compound of Formula 801 and a compound of Formula 802 (such as about 6 equivalents), where X′ is a leaving group such as chloride and R₁ represents an optionally-substituted aryl group such as a halophenyl, is added a catalyst such as AlCl₃ in a solvent such as dichloroethane. The mixture is stirred at about 60° C. for about 2.5 hrs. The resulting product is used directly in step 2, and is converted to a compound of Formula 803 using the proecudure described in Reaction Scheme 1.

Referring to Reaction Scheme 8, Step 3, to a stirred solution of a compound of Formula 803 is added a saturated solution of ammonium chloride followed by iron powder (such as about 8 eq.). The mixture is stirred at room temperature for about 16 hours and the product, a compound of Formula 804, is optionally isolated and purified.

Referring to Reaction Scheme 8, Step 4, the amine of a compound of Formula 804 is further functionalized to yield a compound of Formula 103, where R₇ is an optionally substituted amine.

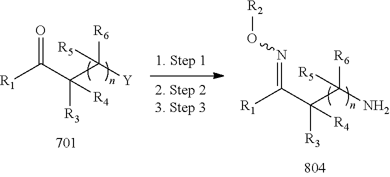

Referring to Reaction Scheme 8b, Step 1, to a stirred solution of a compound of Formula 701, where Y is a leaving group such as chloride or bromide, is added potassium phthalamide (such as about 1.17 eq). The mixture is stirred for about 48 hours. The resulting product is isolated and optionally purified

Referring to Reaction Scheme 8b, Step 2, the product of Step 1 is converted to an oxime, e.g., as described in Reaction Scheme 1.

Referring to Reaction Scheme 8b, Step 3, to a solution of the product of Step 2 is added an excess (such as about 10 eq.) of hydrazine hydrate. The mixture is stirred for about 5 days and the product, a compound of Formula 804, is optionally isolated and purified.

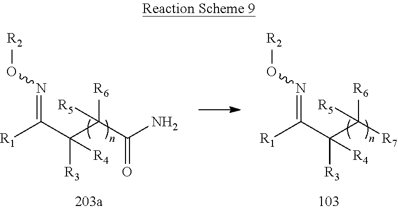

Referring to Reaction Scheme 9, to a mixture of a compound of Formula 203 in THF is added (methoxycarbonylsulfamoyl)triethylammonium hydroxide inner salt. The mixture is stirred for about 2 hours and the product, a compound of Formula 103, where R₇ is cyano, is isolated and optionally purified.

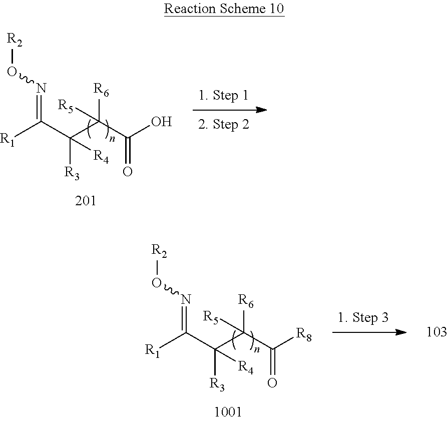

Referring to Reaction Scheme 10, Step 1 to a solution of a carboxylic acid of Formula 201 in a solvent such as DMF is added EDC.HCl (such as about 1 equivalent) and a coupling agent such as HOBt (such as about 1 equivalent). The reaction mixture is stirred at room temperature for about 30 min. N,O,-dimethylhydroxylamine hydrochloride (such as about 1.1 equivalents) is added to the reaction mixture to form the corresponding Weinreb amide. The product is isolated and optionally purified.

Referring to Reaction Scheme 10, Step 2, to a stirred solution of the product of Step 1 in dry THF at a temperature of about −40° C. is added a solution (about 0.5M) of a Grignard reagent, R₈MgBr. The mixture is allowed to warm to room temperature and is stirred for about two hours. The product, a compound of Formula 1001, is isolated and optionally purified.

Referring to Reaction Scheme 10, Step 3, the compound of Formula 1001 is treated, e.g., as described in Reaction Scheme 1, to yield a compound of Formula 103, where R₇ is —C(═N—OR₁₁)R₈.

Provided is a method of inhibiting the catalytic activity of KMO, comprising contacting said KMO with an effective amount of at least one chemical entity described herein.

Also provided is a method of treating a condition or disorder mediated by KMO activity in a subject in need of such a treatment, comprising administering to the subject a therapeutically effective amount of at least one chemical entity described herein.

Also provided is a method of treating a neurodegenerative pathology mediated by KMO activity in a subject in need of such a treatment, comprising administering to the subject a therapeutically effective amount of at least one chemical entity described herein. Also provided is a method for treating disorders mediated by (or at least in part by) the presence of KYNA and/or QUIN. Such diseases include, for example, Huntington's disease and other polyglutamine disorders such as spinocerebellar ataxias, Alzheimer's disease, Parkinson's disease, high-pressure neurological syndrome, dystonia, olivopontocerebellar atrophy, amyotrophic lateral sclerosis, multiple sclerosis, epilepsy, consequences of stroke, cerebral ischemia, hypoxia, multi-infarct dementia, consequences of cerebral trauma or damage, damage to the spinal cord, AIDS-dementia complex, viral or bacterial meningitis, general central nervous system (CNS) infections such as viral, bacterial or parasites, for example, poliomyelitis, Lyme disease (Borrelia burgdorferi infection) and malaria, cancers with cerebral localization, Tourette's syndrome, hepatic encephalopathy, systemic lupus, analgesia and opiate withdrawal symptoms, feeding behavior, schizophrenia, chronic anxiety, depressive disorders, disorders of the developing or aged brain, diabetes, and complications thereof, comprising administering to the subject an effective amount of at least one chemical entity described herein.

Also provided are methods of treatment in which at least one chemical entity described herein is the only active agent given to the subject and also includes methods of treatment in which at least one chemical entity described herein is given to the subject in combination with one or more additional active agents.

In general, the chemical entities described herein will be administered in a therapeutically effective amount by any of the accepted modes of administration for agents that serve similar utilities. The actual amount of the compound, i.e., the active ingredient, will depend upon numerous factors such as the severity ofthe disease to be treated, the age and relative health of the subject, the potency of the compound used, the route and form of administration, and other factors well know to the skilled artisan. The drug can be administered at least once a day, such as once or twice a day.

In some embodiments, the chemical entities described herein are administered as a pharmaceutical composition. Accordingly, provided are pharmaceutical compositions comprising at least one chemical entity described herein, together with at least one pharmaceutically acceptable vehicle chosen from carriers, adjuvants, and excipients.

Pharmaceutically acceptable vehicles must be of sufficiently high purity and sufficiently low toxicity to render them suitable for administration to the animal being treated. The vehicle can be inert or it can possess pharmaceutical benefits. The amount of vehicle employed in conjunction with the chemical entity is sufficient to provide a practical quantity of material for administration per unit dose of the chemical entity.

Exemplary pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; synthetic oils; vegetable oils, such as peanut oil, cottonseed oil, sesame oil, olive oil, and corn oil; polyols such as propylene glycol, glycerine, sorbitol, mannitol, and polyethylene glycol; alginic acid; phosphate buffer solutions; emulsifiers, such as the TWEENS; wetting agents, such sodium lauryl sulfate; coloring agents; flavoring agents; tableting agents; stabilizers; antioxidants; preservatives; pyrogen-free water; isotonic saline; and phosphate buffer solutions.

Optional active agents may be included in a pharmaceutical composition, which do not substantially interfere with the activity of the chemical entity described herein.

Effective concentrations of at least one chemical entity described herein are mixed with a suitable pharmaceutically acceptable vehicle. In instances in which the chemical entity exhibits insufficient solubility, methods for solubilizing compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate.

Upon mixing or addition of a chemical entity described herein, the resulting mixture may be a solution, suspension, emulsion or the like. The form of the resulting mixture depends upon a number of factors, including the intended mode of administration and the solubility of the chemical entity in the chosen vehicle. The effective concentration sufficient for ameliorating the symptoms of the disease treated may be empirically determined.

Chemical entities described herein may be administered orally, topically, parenterally, intravenously, by intramuscular injection, by inhalation or spray, sublingually, transdermally, via buccal administration, rectally, as an ophthalmic solution, or by other means, in dosage unit formulations.

Pharmaceutical compositions may be formulated for oral use, such as for example, tablets, troches, lozenges, aqueous or oily suspensions, dispersible powders or granules, emulsions, hard or soft capsules, or syrups or elixirs. Pharmaceutical compositions intended for oral use may be prepared according to any method known to the art for the manufacture of pharmaceutical compositions and such compositions may contain one or more agents, such as sweetening agents, flavoring agents, coloring agents and preserving agents, in order to provide pharmaceutically elegant and palatable preparations. In some embodiments, oral pharmaceutical compositions contain from 0.1 to 99% of at least one chemical entity described herein. In some embodiments, oral pharmaceutical compositions contain at least 5% (weight %) of at least one chemical entity described herein. Some embodiments contain from 25% to 50% or from 5% to 75% of at least one chemical entity described herein.

Orally administered pharmaceutical compositions also include liquid solutions, emulsions, suspensions, powders, granules, elixirs, tinctures, syrups, and the like. The pharmaceutically acceptable carriers suitable for preparation of such compositions are well known in the art. Oral pharmaceutical compositions may contain preservatives, flavoring agents, sweetening agents, such as sucrose or saccharin, taste-masking agents, and coloring agents.

Typical components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such pharmaceutical compositions may also contain a demulcent.

Chemical entities described herein can be incorporated into oral liquid preparations such as aqueous or oily suspensions, solutions, emulsions, syrups, or elixirs, for example. Moreover, pharmaceutical compositions containing these chemical entities can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monsoleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

For a suspension, typical suspending agents include methylcellulose, sodium carboxymethyl cellulose, AVICEL RC-591, tragacanth and sodium alginate; typical wetting agents include lecithin and polysorbate 80; and typical preservatives include methyl paraben and sodium benzoate.

Aqueous suspensions contain the active material(s) in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents; may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl, or n-propyl p-hydroxybenzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These pharmaceutical compositions may be preserved by the addition of an anti-oxidant such as ascorbic acid.

Pharmaceutical compositions may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

Tablets typically comprise conventional pharmaceutically acceptable adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, can be useful adjuvants for chewable tablets. Capsules (including time release and sustained release formulations) typically comprise one or more solid diluents disclosed above. The selection of carrier components often depends on secondary considerations like taste, cost, and shelf stability.

Such pharmaceutical compositions may also be coated by conventional methods, typically with pH or time-dependent coatings, such that the chemical entity is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Pharmaceutical compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable vehicle, for example as a solution in 1,3-butanediol. Among the acceptable vehicles that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid can be useful in the preparation of injectables.

Chemical entities described herein may be administered parenterally in a sterile medium. Parenteral administration includes subcutaneous injections, intravenous, intramuscular, intrathecal injection or infusion techniques. Chemical entities described herein, depending on the vehicle and concentration used, can either be suspended or dissolved in the vehicle. Advantageously, adjuvants such as local anesthetics, preservatives and buffering agents can be dissolved in the vehicle. In many pharmaceutical compositions for parenteral administration the carrier comprises at least 90% by weight of the total composition. In some embodiments, the carrier for parenteral administration is chosen from propylene glycol, ethyl oleate, pyrrolidone, ethanol, and sesame oil.

Chemical entities described herein may also be administered in the form of suppositories for rectal administration of the drug. These pharmaceutical compositions can be prepared by mixing the drug with a suitable non-irritating excipient that is solid at ordinary temperatures but liquid at rectal temperature and will therefore melt in the rectum to release the drug. Such materials include cocoa butter and polyethylene glycols.

Chemical entities described herein may be formulated for local or topical application, such as for topical application to the skin and mucous membranes, such as in the eye, in the form of gels, creams, and lotions and for application to the eye. Topical pharmaceutical compositions may be in any form including, for example, solutions, creams, ointments, gels, lotions, milks, cleansers, moisturizers, sprays, skin patches, and the like.

Such solutions may be formulated as 0.01%-10% isotonic solutions, pH 5-7, with appropriate salts. Chemical entities described herein may also be formulated for transdermal administration as a transdermal patch.

Topical pharmaceutical compositions comprising at least one chemical entity described herein can be admixed with a variety of carrier materials well known in the art, such as, for example, water, alcohols, aloe vera gel, allantoin, glycerine, vitamin A and E oils, mineral oil, propylene glycol, PPG-2 myristyl propionate, and the like.

Other materials suitable for use in topical carriers include, for example, emollients, solvents, humectants, thickeners and powders. Examples of each of these types of materials, which can be used singly or as mixtures of one or more materials, are as follows:

Representative emollients include stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, and myristyl myristate; propellants, such as propane, butane, iso-butane, dimethyl ether, carbon dioxide, and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate, and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, and ethylene glycol monostearate.

The chemical entities described herein may also be topically administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Other pharmaceutical compositions useful for attaining systemic delivery of the chemical entity include sublingual, buccal and nasal dosage forms. Such pharmaceutical compositions typically comprise one or more of soluble filler substances such as sucrose, sorbitol and mannitol, and binders such as acacia, microcrystalline cellulose, carboxymethyl cellulose, and hydroxypropyl methylcellulose. Glidants, lubricants, sweeteners, colorants, antioxidants and flavoring agents disclosed above may also be included.

Pharmaceutical compositions for inhalation typically can be provided in the form of a solution, suspension or emulsion that can be administered as a dry powder or in the form of an aerosol using a conventional propellant (e.g., dichlorodifluoromethane or trichlorofluoromethane).

The pharmaceutical compositions may also optionally comprise an activity enhancer. The activity enhancer can be chosen from a wide variety of molecules that function in different ways to enhance or be independent of therapeutic effects of the chemical entities described herein. Particular classes of activity enhancers include skin penetration enhancers and absorption enhancers.

Pharmaceutical compositions may also contain additional active agents that can be chosen from a wide variety of molecules, which can function in different ways to enhance the therapeutic effects of at least one chemical entity described herein. These optional other active agents, when present, are typically employed in the pharmaceutical compositions at a level ranging from 0.01% to 15%. Some embodiments contain from 0.1% to 10% by weight of the composition. Other embodiments contain from 0.5% to 5% by weight of the composition.

Also provided are packaged pharmaceutical compositions. Such packaged compositions include a pharmaceutical composition comprising at least one chemical entity described herein, and instructions for using the composition to treat a subject (typically a human patient). In some embodiments, the instructions are for using the pharmaceutical composition to treat a subject suffering a condition or disorder mediated by Kynurenine 3-mono-oxygenase activity. The packaged pharmaceutical composition can include providing prescribing information; for example, to a patient or health care provider, or as a label in a packaged pharmaceutical composition. Prescribing information may include for example efficacy, dosage and administration, contraindication and adverse reaction information pertaining to the pharmaceutical composition.

In all of the foregoing the chemical entities can be administered alone, as mixtures, or in combination with other active agents.

The methods described herein include methods for treating Huntington's disease, including treating memory and/or cognitive impairment associated with Huntington's disease, comprising administering to a subject, simultaneously or sequentially, at least one chemical entity described herein and one or more additional agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone. In methods using simultaneous administration, the agents can be present in a combined composition or can be administered separately. As a result, also provided are pharmaceutical compositions comprising at least one chemical entity described herein and one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone. Similarly, also provided arepackaged pharmaceutical compositions containing a pharmaceutical composition comprising at least one chemical entity described herein, and another composition comprising one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone.

Also provided are methods for treating Parkinson's disease, including treating memory and/or cognitive impairment associated with Parkinson's disease, comprising administering to a subject, simultaneously or sequentially, at least one chemical entity described herein and one or more additional agents used in the treatment of Parkinson's disease such as, but not limited to, Levodopa, Parlodel, Permax, Mirapex, Tasmar, Contan, Kemadin, Artane, and Cogentin. In methods using simultaneous administration, the agents can be present in a combined composition or can be administered separately. Also provided are pharmaceutical compositions comprising at least one chemical entity described herein, and one or more additional pharmaceutical agents used in the treatment of Parkinson's disease, such as, but not limited to, Levodopa, Parlodel, Permax, Mirapex, Tasmar, Contan, Kemadin, Artane, and Cogentin. Also provided are packaged pharmaceutical compositions containing a pharmaceutical composition comprising at least one chemical entity described herein, and another composition comprising one or more additional pharmaceutical agents gent used in the treatment of Parkinson's disease such as, but not limited to, Levodopa, Parlodel, Permax, Mirapex, Tasmar, Contan, Kemadin, Artane, and Cogentin.

Also provided are methods for treating memory and/or cognitive impairment associated with Alzheimer's disease, comprising administering to a subject, simultaneously or sequentially, at least one chemical entity described herein and one or more additional agents used in the treatment of Alzheimer's disease such as, but not limited to, Reminyl, Cognex, Aricept, Exelon, Akatinol, Neotropin, Eldepryl, Estrogen and Cliquinol. In methods using simultaneous administration, the agents can be present in a combined composition or can be administered separately. Also provided are pharmaceutical compositions comprising at least one chemical entity described herein, and one or more additional pharmaceutical agents used in the treatment of Alzheimer's disease such as, but not limited to, Reminyl, Cognex, Aricept, Exelon, Akatinol, Neotropin, Eldepryl, Estrogen and Cliquinol. Similarly, also provided are packaged pharmaceutical compositions containing a pharmaceutical composition comprising at least one chemical entity described herein, and another composition comprising one or more additional pharmaceutical agents used in the treatment of Alzheimer's disease such as, but not limited to Reminyl, Cognex, Aricept, Exelon, Akatinol, Neotropin, Eldepryl, Estrogen and Cliquinol.

Also provided are methods for treating memory and/or cognitive impairment associated with dementia comprising administering to a subject, simultaneously or sequentially, at least one chemical entity and one or more additional agents used in the treatment of dementia such as, but not limited to, Thioridazine, Haloperidol, Risperidone, Cognex, Aricept, and Exelon. In methods using simultaneous administration, the agents can be present in a combined composition or can be administered separately. Also provided are pharmaceutical compositions comprising at least one chemical entity described herein, and one or more additional pharmaceutical agents used in the treatment of dementia such as, but not limited to, Thioridazine, Haloperidol, Risperidone, Cognex, Aricept, and Exelon. Also provided are packaged pharmaceutical compositions containing a pharmaceutical composition comprising at least one chemical entity described herein, and another composition comprising one or more additional pharmaceutical agents used in the treatment of dementia such as, but not limited to, Thioridazine, Haloperidol, Risperidone, Cognex, Aricept, and Exelon.

Also provided are methods for treating memory and/or cognitive impairment associated with epilepsy comprising administering to a subject, simultaneously or sequentially, at least one chemical entity described herein and one or more additional agents used in the treatment of epilepsy such as, but not limited to, Dilantin, Luminol, Tegretol, Depakote, Depakene, Zarontin, Neurontin, Barbita, Solfeton, and Felbatol. In methods using simultaneous administration, the agents can be present in a combined composition or can be administered separately. Also provided are pharmaceutical compositions comprising at least one chemical entity described herein, and one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol. Also provided are packaged pharmaceutical compositions containing a pharmaceutical composition comprising at least one chemical entity described herein, and another composition comprising one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol.

Also provided are methods for treating memory and/or cognitive impairment associated with multiple sclerosis comprising administering to a subject, simultaneously or sequentially, at least one chemical entity described herein and one or more additional agents used in the treatment of multiple sclerosis such as, but not limited to, Detrol, Ditropan XL, OxyContin, Betaseron, Avonex, Azothioprine, Methotrexate, and Copaxone. In methods using simultaneous administration, the agents can be present in a combined composition or can be administered separately. Also provided are pharmaceutical compositions comprising at least one chemical entity described herein, and one or more additional pharmaceutical agents used in the treatment of multiple sclerosis such as, but not limited to, Detrol, Ditropan XL, OxyContin, Betaseron, Avonex, Azothioprine, Methotrexate, and Copaxone. Also provided are packaged pharmaceutical compositions containing a pharmaceutical composition comprising at least one chemical entity described herein, and another composition comprising one or more additional pharmaceutical agents used in the treatment of multiple sclerosis such as, but not limited to, Detrol, Ditropan XL, OxyContin, Betaseron, Avonex, Azothioprine, Methotrexate, and Copaxone.

When used in combination with one or more additional pharmaceutical agent or agents, the described herein may be administered prior to, concurrently with, or following administration of the additional pharmaceutical agent or agents.

The dosages of the compounds described herein depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations.

The chemical entities described herein are typically administered at dosage levels and in a manner customary for KMO inhibitors. For example, the chemical entities can be administered, in single or multiple doses, by oral administration at a dosage level of generally 0.001-100 mg/kg/day, for example, 0.01-100 mg/kg/day, such as 0.1-70 mg/kg/day, for example, 0.5-10 mg/kg/day. Unit dosage forms can contain generally 0.01-1000 mg of at least one chemical entity described herein, for example, 0.1-50 mg of at least one chemical entity described herein. For intravenous administration, the compounds can be administered, in single or multiple dosages, at a dosage level of, for example, 0.001-50 mg/kg/day, such as 0.001-10 mg/kg/day, for example, 0.01-1 mg/kg/day. Unit dosage forms can contain, for example, 0.1-10 mg of at least one chemical entity described herein.

A labeled form of a chemical entity described herein can be used as a diagnostic for identifying and/or obtaining compounds that have the function of modulating an activity of KMO as described herein. The chemical entities described herein may additionally be used for validating, optimizing, and standardizing bioassays.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In carrying out the procedures of the methods described herein, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

### EXAMPLES

The chemical entities, compositions, and methods described herein are further illustrated by the following non-limiting examples.

As used herein, the following abbreviations have the following meanings. If an abbreviation is not defined, it has its generally accepted meaning.
CDI=carbonyldiimidazole
DCM=dichloromethane
DME=dimethyl ether
DMEM=Dulbecco's modified Eagle's medium
DMF=N,N-dimethylformamide
DMSO=dimethylsulfoxide
EDC.HCl=1-Ethyl-3-(3-dimethylaminopropyl)carbodiimide hydrochloride
EtOH=ethanol
Et₂O=diethylether
EtOAc=ethyl acetate
g=gram
hr=hour
hrs=hours
HOBt=tert-butyl alcohol
LiHMDS=lithium hexamethyl-disilazide
LC/MS=liquid chomatography/mass spectrometry
mg=milligram
min=minutes
mL=milliliter
mmol=millimoles
mM=millimolar
ng=nanogram
nm=nanometer
nM=nanomolar
PBS=phosphate buffered saline
rt=room temperature
TBME=t-butyl methyl ether
THF=tetrahydrofuran
TMOF=trimethylorthoformate
μL=microliter
μM=micromolar

### Example 1

### Preparation of 1-(3,4-dichlorophenyl)-3-(3-methyl-1,2,4-oxadiazol-5-yl)propan-1-one O-methyl oxime

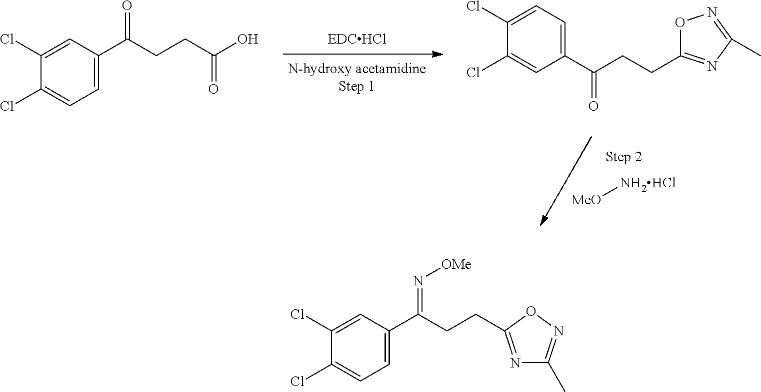

Step 1

To a stirred solution of 4-(3,4-dichloro-phenyl)-4-oxo-butyric acid (1 eq) in DMF was added EDC.HCl (1 eq) and HOBt (1 eq). The reaction mixture was stirred at rt for 25 min. N-hydroxy acetamidine was added and stirring continued for 2 hrs. Triethylamine (1.5 eq) was added and the reaction mixture stirred at 80° C. for 16 hrs. After cooling, water (60 vol) was added and the solution extracted with EtOAc (3×60 vol). The combined organic layers were washed with water (60 vol), saturated sodium chloride (60 vol), dried over MgSO₄, filtered, and the solvent removed in vacuo to give crude product. Purification by flash column chromatography (eluent: 0.5% MeOH in DCM) gave 1-(3,4-dichloro-phenyl)-3-(3-methyl-[1,2,4]oxadiazol-5-yl)-propan-1-one.

Step 2

To a stirred solution of 1-(3,4-dichloro-phenyl)-3-(3-methyl-[1,2,4]oxadiazol-5-yl)-propan-1-one (1 eq) in EtOH was added O-methylhydroxylamine hydrochloride (5 eq) and pyridine (3.3 eq). The reaction mixture was stirred at 95° C. and monitored by LCMS. After cooling, the reaction mixture was concentrated in vacuo to give a white solid. Water was added and the resulting mixture was extracted with DCM (3×). The combined organic layers were washed with 2M citric acid, saturated aqueous NaCl, dried over MgSO₄, filtered, and the solvent removed in vacuo to give the crude product. The crude product was purified by flash column chromatography to yield 1-(3,4-dichlorophenyl)-3-(3-methyl-1,2,4-oxadiazol-5-yl)propan-1-one O-methyl oxime.

### Example 2

### Preparation of 1-(3,4-dichlorophenyl)-4-(1H-imidazol-1-yl)butan-1-one O-methyloxime

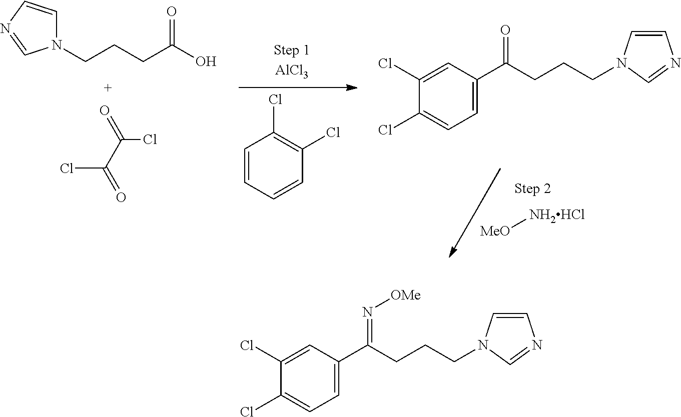

Step 1

To a stirred suspension of 4-(1H-imidazol-1-yl)butanoic acid (1 eq) in DCM (10 vol) was added oxalyl chloride (2M solution in DCM, 2 eq) and a drop of DMF. The reaction mixture was allowed to stir at rt for 4.5 hrs. The reaction mixture was concentrated in vacuo and to the residue was added 1,2-di-chlorobenzene (1 eq), AlCl₃ (3 eq), and dichloroethane. The reaction mixture was heated at 60° C. for 2 hrs. After cooling down, the reaction mixture was poured onto ice and the resulting aqueous layer washed with DCM (20 vol). The aqueous was then basified with 2M NaOH and extracted with DCM (2×20 vol). The combined organic layers were dried over MgSO₄, filtered, and the solvent removed in vacuo to give 1-(3,4-dichloro-phenyl)-4-imidazol-1-yl-butan-1-one.

Step 2

To a stirred solution of 1-(3,4-dichloro-phenyl)-4-imidazol-1-yl-butan-1-one (1 eq) was added O-methylhydroxylamine hydrochloride (5 eq) and pyridine (3.3 eq). The reaction mixture was stirred at 95° C. and monitored by LCMS. After cooling, the reaction mixture was concentrated in vacuo to give a white solid. Water was added and the resulting mixture was extracted with DCM (3×). The combined organic layers were washed with 2M citric acid, saturated aqueous NaCl, dried over MgSO₄, filtered, and the solvent removed in vacuo to give the crude product. The crude product was purified by flash column chromatography to yield 1-(3,4-dichlorophenyl)-4-(1H-imidazol-1-yl)butan-1-one O-methyl oxime.

### Example 3

### Preparation of 1-(3,4-Dichloro-phenyl)-3-imidazol-1-yl-propan-1-one O-methyl-oxime

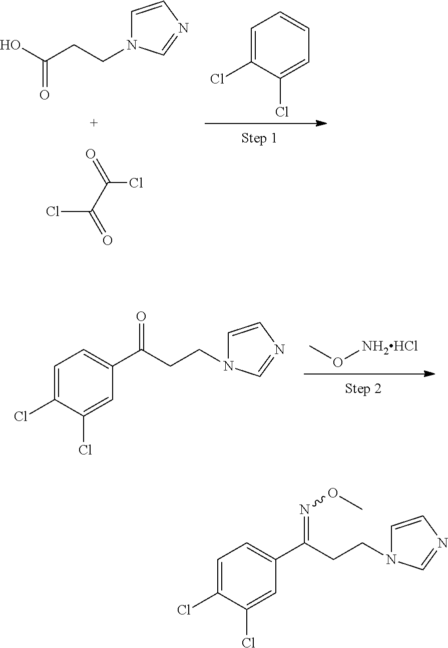

Step 1

To a stirred suspension of 4-(1H-imidazol-1-yl)propanoic acid (1 eq) in DCM (10 vol) was added oxalyl chloride (2M solution in DCM, 2 eq) and a drop of DMF. The reaction mixture was allowed to stir at rt for 4.5 hrs. The reaction mixture was concentrated in vacuo and to the residue was added 1,2-di-chlorobenzene (1 eq), AlCl₃ (3 eq), and dichloroethane. The reaction mixture was heated at 60° C. for 2 hrs. After cooling down, the reaction mixture was poured onto ice and the resulting aqueous layer washed with DCM (20 vol). The aqueous phase was then basified with 2M NaOH and extracted with DCM (2×20 vol). The combined organic layers were dried over MgSO₄, filtered, and the solvent removed in vacuo to give 1-(3,4-dichloro-phenyl)-4-imidazol-1-yl-propan-1-one.

Step 2

To a stirred solution of 1-(3,4-dichloro-phenyl)-4-imidazol-1-yl-propan-1-one (1 eq) was added O-methylhydroxylamine hydrochloride (5 eq) and pyridine (3.3 eq). The reaction mixture was stirred at 95° C. and monitored by LCMS. After cooling, the reaction mixture was concentrated in vacuo to give a white solid. Water was added and the resulting mixture was extracted with DCM (3×). The combined organic layers were washed with 2M citric acid, saturated aqueous NaCl, dried over MgSO₄, filtered, and the solvent removed in vacuo to give the crude product. The crude product was purified by flash column chromatography to yield 1-(3,4-dichloro-phenyl)-3-imidazol-1-yl-propan-1-one O-methyl-oxime.

### Example 4

### Preparation of 1-(3,4-Dichloro-phenyl)-3-(2H-tetrazol-5-yl)-propan-1-one O-methyl-oxime

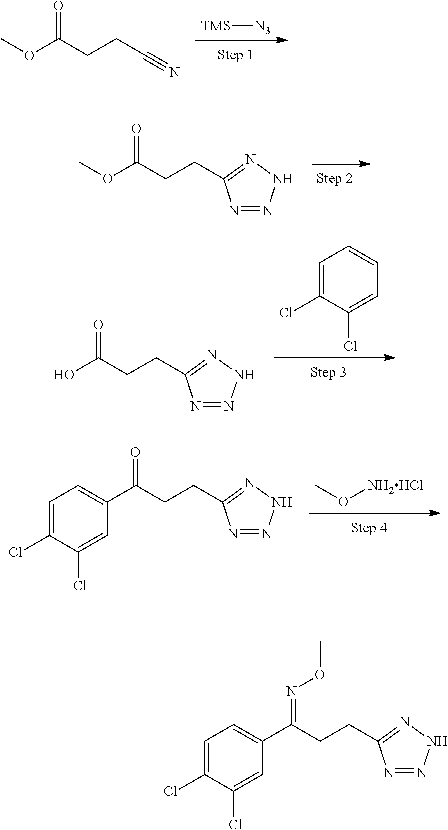

Step 1

To a stirred solution of methyl 3-cyanopropionate (1 eq) in toluene (25 vol) was added trimethylsilylazide (2 eq) and di-butyl tin oxide (0.1 eq) and the reaction mixture was refluxed for 16 hrs. The reaction mixture was concentrated in vacuo, the residue dissolved in MeOH (50 vol) and concentrated. The residue was dissolved in EtOAc (60 vol) and washed with saturated NaHCO₃ (60 vol). The combined aqueous layers were acidified with conc HCl and extracted with EtOAc (2×60 vol). The combined organic layers were washed with saturated sodium chloride (60 vol), dried over MgSO₄, filtered and solvent removed in vacuo to give 3-(2H-tetrazol-5-yl)-propionic acid methyl ester.

Step 2

To a stirred solution of 3-(2H-tetrazol-5-yl)-propionic acid methyl ester (1 eq) in MeOH (15 vol) was added NaOH (2 eq) dissolved in water (2 vol). The reaction mixture was stirred at rt for 2 hrs. A further 2 eq of NaOH was added and the reaction stirred for an additional 2 hrs. The reaction mixture was concentrated in vacuo and the resultant solids dissolved in water (4 vol), acidified with concentrated HCl and extracted with DCM (2×60 vol). The combined organic layers were dried over MgSO₄, filtered and solvent removed in vacuo to give 3-(2H-tetrazol-5-yl)-propionic acid as a brown solid.

Step 3

To a stirred suspension of 3-(2H-tetrazol-5-yl)-propionic acid (1 eq) was added oxalyl chloride (31.3 vol) and the reaction mixture stirred at 60° C. for 30 min before being concentrated in vacuo. The residue was dissolved in 1,2-di-chlorobenzene (6 eq) and AlCl₃ (3 eq). The reaction mixture was heated at 60° C. for 1.5 hrs. After cooling, the mixture was poured onto ice and the resultant solid filtered and washed with heptane (80 vol) followed by DCM (40 vol). Purification by flash column chromatography (eluent: 2% MeOH in DCM) followed by hot filtration from EtOAc/Heptane yielded 1-(3,4-dichloro-phenyl)-3-(2H-tetrazol-5-yl)-propan-1-one.

Step 4

To a stirred solution of 1-(3,4-dichloro-phenyl)-3-(2H-tetrazol-5-yl)-propan-1-one (1 eq) was added O-methylhydroxylamine hydrochloride (5 eq) and pyridine (3.3 eq). The reaction mixture was stirred at 95° C. and monitored by LCMS. After cooling, the reaction mixture was concentrated in vacuo to give a white solid. Water was added and the resulting mixture was extracted with DCM (3×). The combined organic layers were washed with 2M citric acid, saturated aqueous NaCl, dried over MgSO₄, filtered, and the solvent removed in vacuo to give the crude product. The crude product was purified by flash column chromatography to yield 1-(3,4-dichloro-phenyl)-3-(2H-tetrazol-5-yl)-propan-1-one O-methyl-oxime.

### Example 5

### Preparation of methyl 1-(3,4-dichlorophenyl)-5-methoxypentan-1-one O-methyl oxime

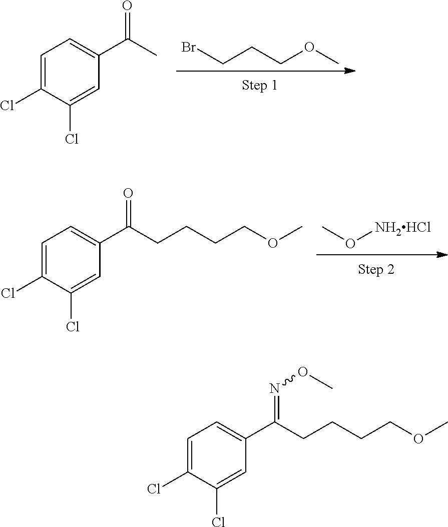

Step 1

A stirred solution of 1-(3,4-dichlorophenyl)-ethanone (1 eq) in THF (10 vol) was cooled to −55° C. 1M LiHMDS (1.1 eq) was added dropwise before cooling the reaction mixture to −78° C. 1-Bromo-3-methoxypropane (1 eq) was added dropwise. The reaction mixture was allowed to warm to rt and stirred for 96 hrs. The reaction was quenched with 1M HCl and extracted with TBME (3×10 vol). The combined organic layers were washed with saturated sodium chloride (20 vol), dried over MgSO₄, filtered and the solvent removed in vacuo to give the crude product. Purification by flash column chromatography (eluent: [9:1] heptane:EtOAc) yielded 1-(3,4-dichloro-phenyl)-5-methoxy-pentan-1-one.

Step 2

To a stirred solution of 1-(3,4-dichloro-phenyl)-5-methoxy-pentan-1-one (1 eq) in EtOH was added O-methylhydroxylamine hydrochloride (5 eq) and pyridine (3.3 eq). The reaction mixture was stirred at 95° C. and monitored by LCMS until completion. After cooling, the reaction mixture was concentrated in vacuo to give a white solid. Water was added and the resulting mixture was extracted with DCM (3×). The combined organic layers were washed with 2M citric acid, saturated aqueous NaCl, dried over MgSO₄, filtered, and the solvent removed in vacuo to give the crude product. The crude product was purified by flash column chromatography to yield 1-(3,4-dichlorophenyl)-5-methoxypentan-1-one O-methyl oxime.

### Example 6

### Preparation of 4-(3,4-Dichloro-phenyl)-4-methoxyimino-N-pyrrolidin-3-yl-butyramide

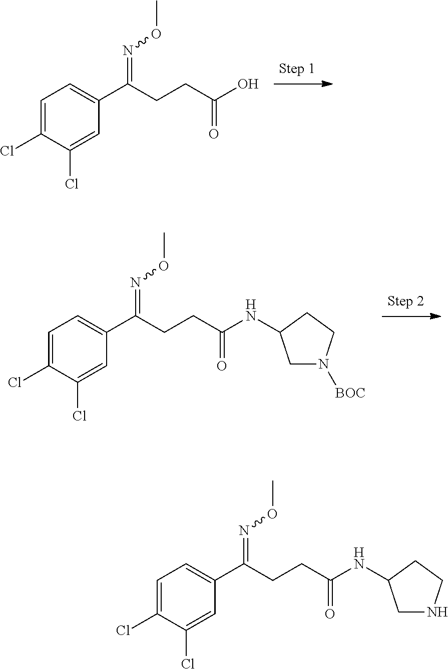

Step 1

To a solution of 4-(3,4-dichlorophenyl)-4-(methoxyimino)butanoic acid (1 eq) in DMF was added EDC.HCl (1 eq) and HOBt (1 eq). The reaction mixture was stirred at ambient temperature for 30 minutes after which time tert-butyl 3-aminopyrrolidine-1-carboxylate (3 eq) was added. The reaction mixture was heated at 50° C. and the reaction was monitored by LCMS to completion whereupon the reaction mixture was allowed to cool to room temperature and poured into water (100 vol). The reaction mixture was extracted with EtOAc (100 vol) and the organic layer was washed with a 0.25M solution of potassium carbonate (3×100 vol), water (100 vol), saturated aqueous NaCl (2×100 vol) and the solvent removed in vacuo to afford the crude product. The crude product was re-dissolved in EtOAc, washed with 0.5M citric acid (3×100 vol), saturated NaCl (2×100 vol), dried MgSO₄ and concentrated. The product was triturated with DCM and then dried in vacuo to yield 3-[4-(3,4-Dichloro-phenyl)-4-methoxyimino-butyrylamino]-pyrrolidine-1-carboxylic acid tert-butyl ester.

Step 2

3-[4-(3,4-Dichloro-phenyl)-4-methoxyimino-butyrylamino]-pyrrolidine-1-carboxylic acid tert-butyl ester (1 eq) was treated with DCM/TFA (30 vol/30 vol) at 0° C., then stirred at ambient temperature for 1 hour. The solvents were removed in vacuo and the residue evaporated with DCM (×3). Water was added and the aqueous basified to pH 10-11 with 1M sodium hydroxide. The aqueous was extracted with DCM, washed with water (3×100 vol), brine (2×100 vol), dried MgSO₄, and then evaporated in vacuo to yield 4-(3,4-Dichloro-phenyl)-4-methoxyimino-N-pyrrolidin-3-yl-butyramide

### Example 7

### Preparation of 4-(3,4-Dichloro-phenyl)-4-methoxyimino-butyronitrile

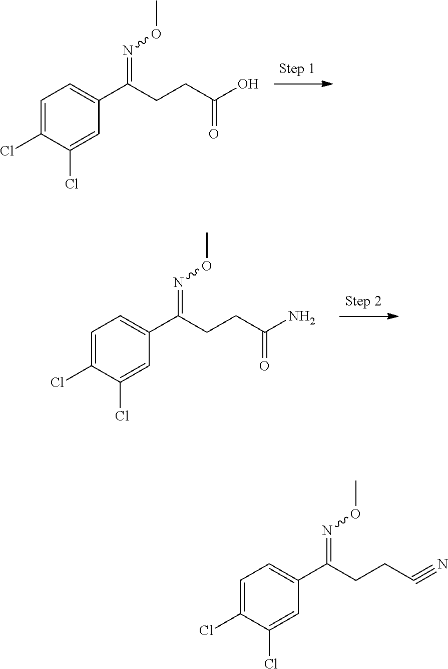

Step 1

To a stirred solution of 4-(3,4-dichloro-phenyl)-4-methoxyimino-butanoic acid in THF (10 vol) was added 1,1′-carbonyldiimidazole (1.1 eq). The solution was stirred at room temperature for 30 min, after which time a solution of ammonia (0.5 M) in 1,4-dioxane (18 eq) was added. The reaction mixture was stirred at room temperature and monitored by LCMS. The solvent was removed in vacuo and the residue dissolved in DCM (40 vol) and washed twice with a saturated solution of NaHCO₃, dried over Na₂SO₄ and filtered. The solvent was removed in vacuo to afford the crude material as a solid, which was further purified by trituration with diethyl ether to yield 4-(3,4-dichloro-phenyl)-4-methoxyimino-butyramide.

Step 2

4-(3,4-dichloro-phenyl)-4-methoxyimino-butyramide was dissolved in THF (1.2 vol) and (methoxycarbonylsulfamoyl)triethylammonium hydroxide inner salt was added. The reaction mixture was stirred at ambient temperature for 2 hours. The solvent was removed in vacuo and the residue dissolved in DCM (20 vol) and washed with water (2×20 vol), dried over Na₂SO₄ and filtered. The solvent was removed in vacuo to afford the crude material as an orange oil, which was further purified by flash column chromatography to yield 4-(3,4-Dichloro-phenyl)-4-methoxyimino-butyronitrile.

### Example 8

### Preparation of 1-(3,4-dichloro-phenyl)-3-(1H-imidazo[4,5-c]pyridin-2-yl)-propan-1-one o-methyl-oxime

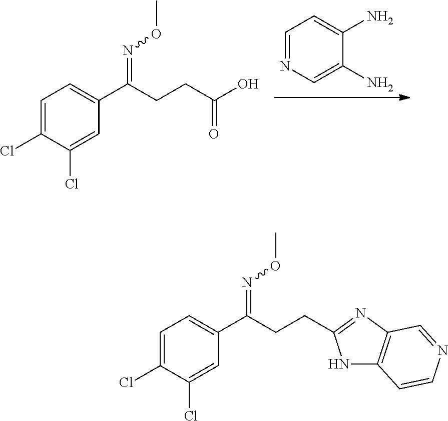

To a solution of 4-(3,4-dichloro-phenyl)-4-methoxyimino-butanoic acid (1 eq) in DMF (20 vol) was added EDC.HCl (1.1 eq). The reaction mixture was stirred at ambient temperature for 30 minutes after which time 3,4-diaminopyridine (1.1 eq) was added. The reaction was monitored by LCMS. After completion a saturated solution of NaHCO₃ (20 vol) was added and the compound was extracted with EtOAc (2×20 vol), dried with MgSO₄, filtered and the solvent was removed in vacuo to yield an amide intermediate, which was dissolved in acetic acid (10 vol) and submitted to microwave irradiation for 1 hour at 100° C. The acetic acid was removed in vacuo and the residue was purified by flash column chromatography (eluent: 2% MeOH in DCM to 4% MeOH in DCM to yield 1-(3,4-dichloro-phenyl)-3-(1H-imidazo[4,5-c]pyridin-2-yl)-propan-1-one o-methyl-oxime.

### Example 9

### Preparation of 1-(3,4-dichloro-phenyl)-3-(5-methyl-[1,2,4]oxadiazol-3-yl)-propan-1-one O-methyl-oxime

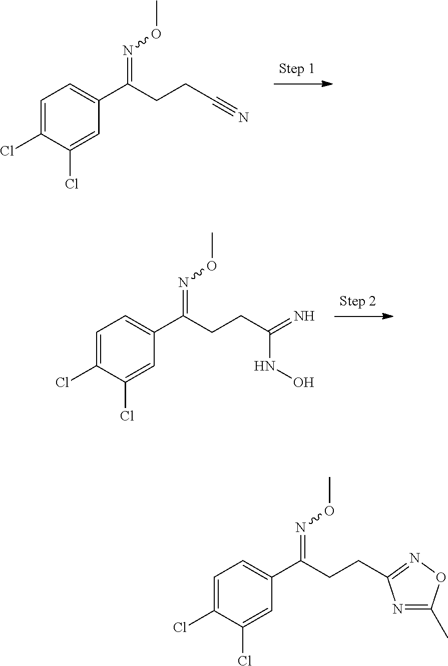

Step 1

To a stirred solution of 4-(3,4-dichloro-phenyl)-4-methoxyimino-butyronitrile (1 eq) in ethanol (20 vol) was added hydroxylamine sulfate (3 eq) followed by an aqueous solution of sodium carbonate (3 eq) (20 vol), and the reaction mixture was stirred at 70° C. for 11 hours. Ethanol was removed in vacuo and the remaining aqueous solution was neutralized to pH 7, extracted with EtOAc (60 vol), dried over MgSO₄, filtered and the solvent removed in vacuo to afford a pale yellow glassy solid, which was further purified by flash column chromatography (eluent: [1:4] to [1:1] EtOAc:heptane) to yield 4-(3,4-dichloro-phenyl)-N-hydroxy-4-methoxyimino-butyramidine.

Step 2

To a stirred solution of 4-(3,4-dichloro-phenyl)-N-hydroxy-4-methoxyimino-butyramidine (1 eq) in DCM (40 vol) was added triethylamine (1.1 eq), followed by acetyl chloride (1.1 eq) and the reaction mixture was stirred at ambient temperature and monitored by LCMS. The solution was washed with water (10 vol), 10% citric acid solution (10 vol), saturated NaHCO₃ solution (10 vol) and brine (10 vol). It was dried over MgSO₄, filtered and the solvent removed in vacuo. The residue was dissolved in pyridine (20 vol) and heated to 115° C. while stirring for 6 hours. Pyridine was removed in vacuo and DCM was added. The organic layer was washed with 10% citric acid solution (10 vol), dried over MgSO₄, filtered and the solvent removed in vacuo. The crude material was purified by flash column chromatography (eluent: [1:2] EtOAc:heptane) to yield 1-(3,4-dichloro-phenyl)-3-(5-methyl-[1,2,4]oxadiazol-3-yl)-propan-1-one O-methyl-oxime.

### Example 10

### Preparation of N-[3-(3,4-Dichloro-phenyl)-3-methoxyimino-propyl]-methanesulfonamide

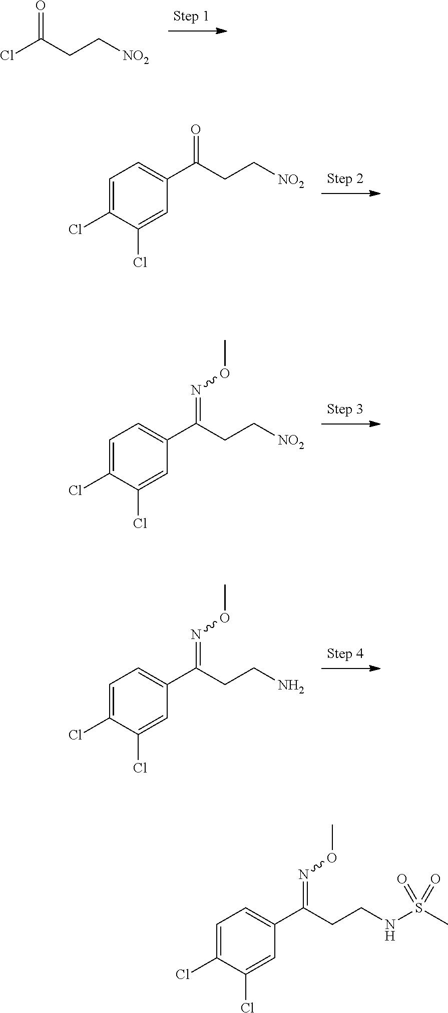

Step 1

To a stirred suspension of 3-nitropropionic acid chloride (1 eq) in 1,2-di-chlorobenzene (6 eq) was added aluminium chloride (3 eq). The reaction mixture was heated at 60° C. for 2.5 hours. After cooling, the reaction mixture was poured onto ice whereupon heptane was added and the mixture stirred for 30 minutes. The resulting solids were filtered and washed with water (×2) and heptane (×2) to yield 1-(3,4-dichloro-phenyl)-3-nitro-propan-1-one.

Step 2

To a stirred solution of 1-(3,4-dichloro-phenyl)-3-nitro-propan-1-one (1 eq) in EtOH was added O-methylhydroxylamine hydrochloride (5 eq) and pyridine (3.3 eq). The reaction mixture was stirred at 95° C. and monitored by LCMS. After cooling, the reaction mixture was concentrated in vacuo to give a solid. Water was added and the resulting mixture was extracted with DCM (×3). The combined organic layers were washed with 2M citric acid, saturated aqueous NaCl, dried over MgSO₄ or Na₂SO₄, filtered, and the solvent removed in vacuo to the give crude product which was purified by flash column chromatography to yield 1-(3,4-dichloro-phenyl)-3-nitro-propan-1-one o-methyl-oxime.

Step 3

A saturated solution of ammonium chloride (10 vol) was added to a stirred solution of 1-(3,4-dichloro-phenyl)-3-nitro-propan-1-one o-methyl-oxime in a mixture of water/ethanol (10 vol/30 vol). Iron powder (8 eq) was added to the reaction mixture, which was then stirred at ambient temperature for 16 hrs. The reaction mixture was then filtered through celite, which was washed with EtOAc. The solvents were evaporated in vacuo to afford 3-Amino-1-(3,4-dichloro-phenyl)-propan-1-one o-methyl-oxime as an orange oil, which was used in step 4 without further purification.

Step 4

3-Amino-1-(3,4-dichloro-phenyl)-propan-1-one o-methyl-oxime (1 eq) was reacted in pyridine (2 vol) with methanesulfonyl chloride (2 eq). The reaction mixture was stirred at room temperature and monitored by LCMS. The reaction was quenched with water and extracted using EtOAc. The organic layer was washed with water (×3) and with brine (×3), dried with MgSO₄, filtered and the solvent was removed in vacuo to afford an oil, which was further purified by flash column chromatography (eluent: [0:100] to [80:20] EtOAc:heptane) to yield N-{3-(3,4-Dichloro-phenyl)-3-methoxyimino-propyl}-methanesulfonamide.

### Example 11

### Preparation of 4-(3,4-dichloro-phenyl)-4-methoxyimino-butyric acid (S)-2-(R)-3,4-dihydroxy-5-oxo-2,5-dihydro-furan-2-yl)-2-hydroxy-ethyl ester

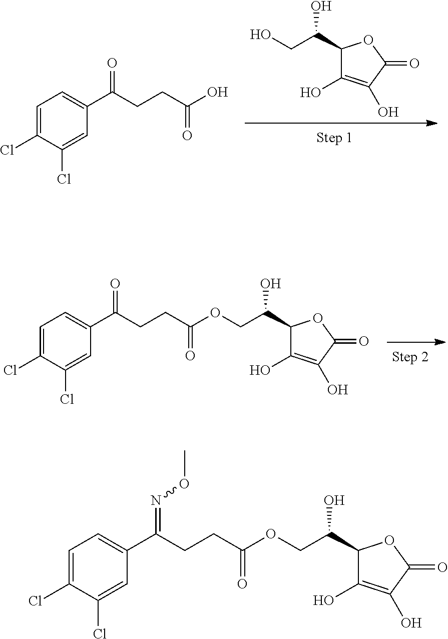

Step 1

To a stirred solution of 4-(3,4-dichloro-phenyl)-4-oxo-butyric acid (1 eq) in sulfuric acid (95%) (15 vol) was added ascorbic acid (1 eq) and the reaction mixture was stirred at 60° C. for 1 hour. The reaction mixture was cooled to room temperature and poured onto ice. The organics were extracted using EtOAc, washed with brine and evaporated in vacuo. Purification was achieved by flash column chromatography (eluent: DCM to 5% MeOH in DCM) to yield (S)-2-((R)-3,4-dihydroxy-5-oxo-2,5-dihydrofuran-2-yl)-2-hydroxyethyl 4-(3,4-dichlorophenyl)-4-oxobutanoate.

Step 2

To a stirred solution of (S)-2-((R)-3,4-dihydroxy-5-oxo-2,5-dihydrofuran-2-yl)-2-hydroxyethyl 4-(3,4-dichlorophenyl)-4-oxobutanoate (1 eq) in EtOH was added O-methylhydroxylamine hydrochloride (5 eq) and pyridine (3.3 eq). The reaction mixture was stirred at 95° C. and monitored by LCMS. After cooling, the reaction mixture was concentrated in vacuo to give a solid. Water was added and the resulting mixture was extracted with DCM (×3). The combined organic layers were washed with 2M citric acid, saturated aqueous NaCl, dried over MgSO₄ or Na₂SO₄, filtered, and the solvent removed in vacuo to the give crude product which was purified by flash column chromatography to yield 4-(3,4-dichloro-phenyl)-4-methoxyimino-butyric acid (S)-2-(R)-3,4-dihydroxy-5-oxo-2,5-dihydro-furan-2-yl)-2-hydroxy-ethyl ester.

### Example 12

### Preparation of 4-(3,4-dichlorophenyl)-4-(2-(dimethylamino)ethoxyimino)butanoic acid

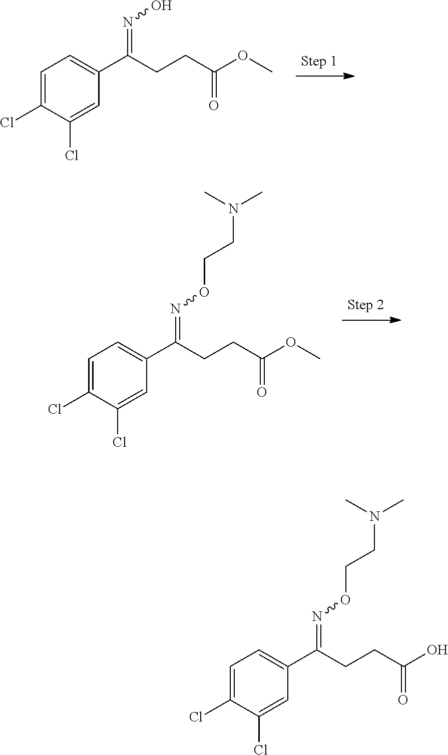

Step 1

To a stirred solution of 4-(3,4-dichloro-phenyl)-4-hydroxyimino-butyric acid methyl ester (1 eq) in DMF (11 vol) was added sodium hydride (2.1 eq) and the reaction mixture was stirred under nitrogen for 20 min. To the reaction mixture was slowly added a suspension of N,N-dimethylaminoethylbromide hydromide (1.1 eq) in DMF and then stirred at ambient temperature for 2 hours. The reaction mixture was then repeatedly heated (maximum of 100° C.) and stirred at ambient temperature for various times over several days until a 50% conversion of product was monitored by LCMS. The reaction mixture was then diluted with water and extracted with EtOAc. The organic layer was washed with brine, dried over MgSO₄, filtered and the solvent removed in vacuo. The crude material was purified by prep HPLC to yield methyl 4-(3,4-dichlorophenyl)-4-(2-(dimethylamino)ethoxyimino)butanoate.

Step 2

To a stirred solution of methyl 4-(3,4-dichlorophenyl)-4-(2-(dimethylamino) ethoxyimino)butanoate (1 eq) in MeOH or THF is added 1M NaOH solution. The reaction mixture is stirred at room temperature and monitored by LCMS. After completion the reaction mixture is concentrated in vacuo and the residue dissolved in water, acidified with conc HCl and extracted with DCM (×3). The combined organic layers are dried over MgSO₄, filtered and the solvent removed in vacuo to yield 4-(3,4-dichlorophenyl)-4-(2-(dimethylamino)ethoxyimino)butanoic acid.

### Example 13

Using procedures similar to those described herein, the following compounds were prepared.

| Structure | Name | [M + H]⁺ |
|---|---|---|
| 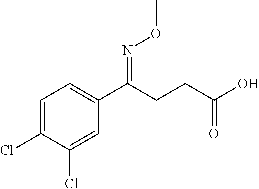 | 4-(3,4-Dichloro-phenyl)-4- methoxyimino-butyric acid | 276 |
| 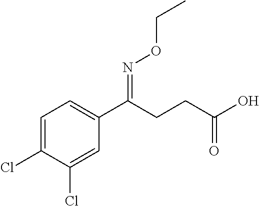 | 4-(3,4-Dichloro-phenyl)-4- ethoxyimino-butyric acid | 290 |
| 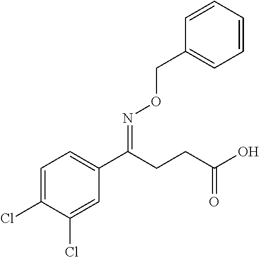 | 4-Benzyloxyimino-4-(3,4- dichloro-phenyl)-butyric acid | 352 |
| 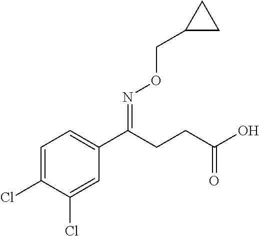 | 4-Cyclopropylmethoxyimino- 4-(3,4-dichloro-phenyl)- butyric acid | 316 |
| 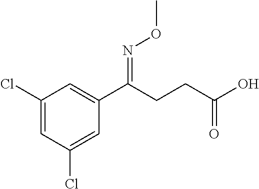 | 4-(3,5-Dichloro-phenyl)-4- methoxyimino-butyric acid | 276 |
| 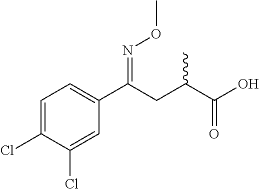 | 4-(3,4-Dichloro-phenyl)-4- methoxyimino-2-methyl- butyric acid | 290 |
| 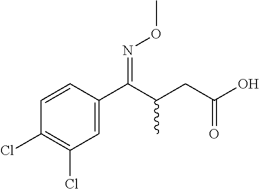 | 4-(3,4-Dichloro-phenyl)-4- methoxyimino-3-methyl- butyric acid | 290 |
| 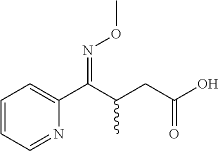 | 4-Methoxyimino-4-pyridin-2- yl-butyric acid | 209 |
| 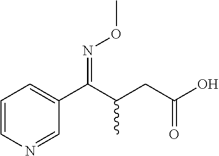 | 4-Methoxyimino-4-pyridin-3- yl-butyric acid | 209 |
| 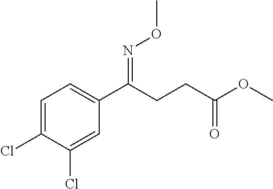 | 4-(3,4-Dichloro-phenyl)-4- methoxyimino-butyric acid methyl ester | 290 |
| 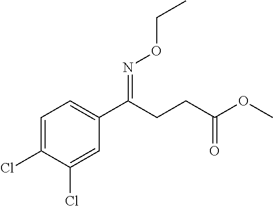 | 4-(3,4-Dichloro-phenyl)-4- ethoxyimino-butyric acid methyl ester | 304 |
| 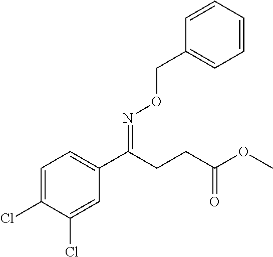 | 4-Benzyloxyimino-4-(3,4- dichloro-phenyl)-butyric acid methyl ester | 366 |
| 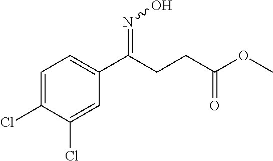 | 4-(3,4-Dichloro-phenyl)-4- hydroxyimino-butyric acid methyl ester | 276 |
| 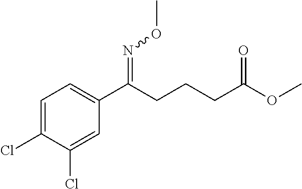 | 5-(3,4-Dichloro-phenyl)-5- methoxyimino-pentanoic acid methyl ester | 304 |
| 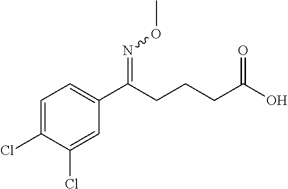 | 5-(3,4-Dichloro-phenyl)-5- methoxyimino-pentanoic acid | 290 |
| 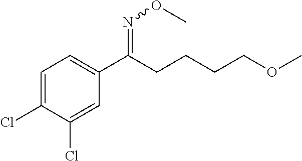 | 1-(3,4-Dichloro-phenyl)-5- methoxy-pentan-1-one O- methyl-oxime | 290 |
| 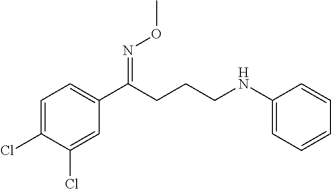 | 4-(3,4-Dichloro-phenyl)-4- methoxyimino-N-phenyl- butyramide | 351 |
| 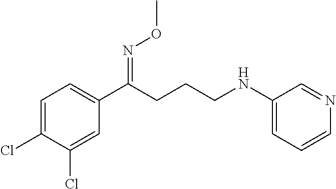 | 4-(3,4-Dichloro-phenyl)-4- methoxyimino-N-pyridin-3- yl-butyramide | 352 |
| 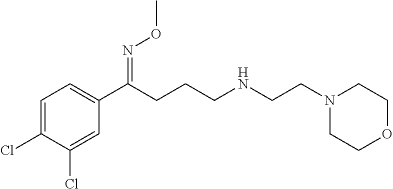 | 4-(3,4-Dichloro-phenyl)-4- methoxyimino-N-(2- morpholin-4-yl-ethyl)- butyramide | 388 |
| 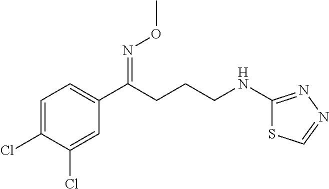 | 4-(3,4-Dichloro-phenyl)-4- methoxyimino-N- [1,3,4]thiadiazol-2-yl- butyramide | 359 |
| 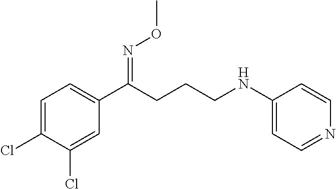 | 4-(3,4-Dichloro-phenyl)-4- methoxyimino-N-pyridin-4- yl-butyramide | 352 |
| 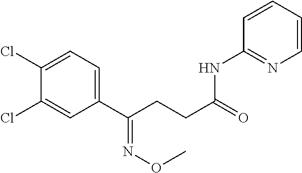 | 4-(3,4-Dichloro-phenyl)-4- methoxyimino-N-pyridin-2- yl-butyramide | 352 |
| 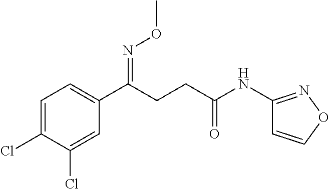 | 4-(3,4-Dichloro-phenyl)-N- isoxazol-3-yl-4- methoxyimino-butyramide | 342 |
| 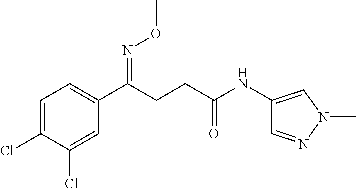 | 4-(3,4-Dichloro-phenyl)-4- methoxyimino-N-(1-methyl- 1H-pyrazol-4-yl)-butyramide | 355 |
| 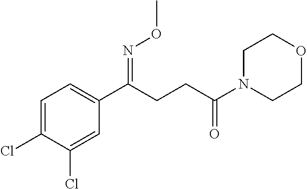 | 1-(3,4-Dichloro-phenyl)-4- morpholin-4-yl-butane-1,4- dione 1-(O-methyl-oxime) | 345 |
| 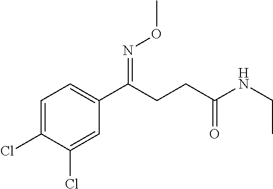 | 4-(3,4-Dichloro-phenyl)-N- ethyl-4-methoxyimino- butyramide | 303 |
| 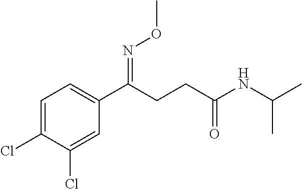 | 4-(3,4-Dichloro-phenyl)-N- isopropyl-4-methoxyimino- butyramide | 317 |
| 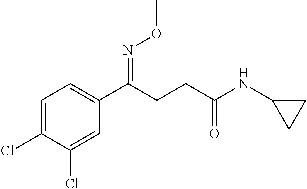 | N-Cyclopropyl-4-(3,4- dichloro-phenyl)-4- methoxyimino-butyramide | 315 |
| 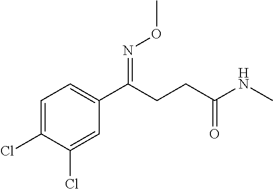 | 4-(3,4-Dichloro-phenyl)-4- methoxyimino-N-methyl- butyramide | 289 |
| 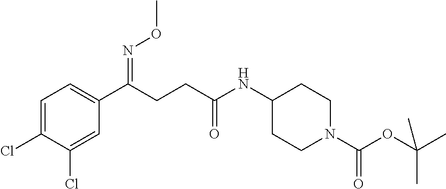 | 4-[4-(3,4-Dichloro-phenyl)-4- methoxyimino- butyrylamino]-piperidine-1- carboxylic acid tert-butyl ester | 458 |
| 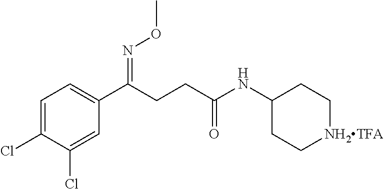 | 4-[4-(3,4-Dichloro-phenyl)-4- methoxyimino- butyrylamino]-piperidinium trifluoroacetate | 358 |
| 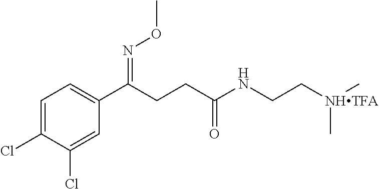 | {2-[4-(3,4-Dichloro-phenyl)- 4-methoxyimino- butyrylamino]-ethyl}- dimethyl-ammonium trifluoroacetate | 346.19 |
| 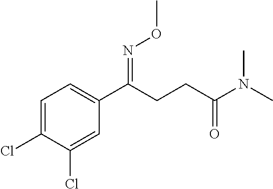 | 4-(3,4-Dichloro-phenyl)-4- methoxyimino-N,N-dimethyl- butyramide | 303 |
| 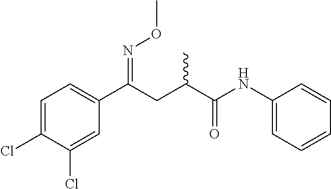 | 4-(3,4-Dichloro-phenyl)-4- methoxyimino-2-methyl-N- phenyl-butyramide | 365 |
| 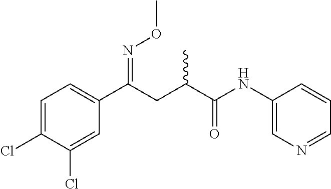 | 4-(3,4-Dichloro-phenyl)-4- methoxyimino-2-methyl-N- pyridin-3-yl-butyramide | 366 |
| 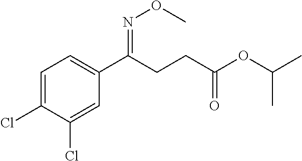 | 4-(3,4-Dichloro-phenyl)-4- methoxyimino-butyric acid isopropyl ester | 318 |
| 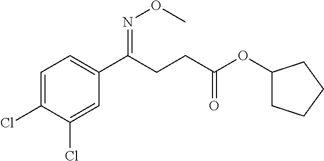 | 4-(3,4-Dichloro-phenyl)-4- methoxyimino-butyric acid cyclopentyl ester | 344 |
| 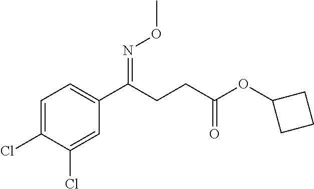 | 4-(3,4-Dichloro-phenyl)-4- methoxyimino-butyric acid cyclobutyl ester | 330 |
| 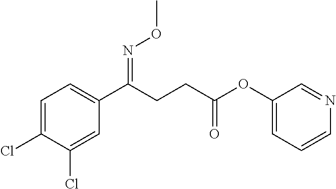 | 4-(3,4-Dichloro-phenyl)-4- methoxyimino-butyric acid pyridin-3-yl ester | 353 |
| 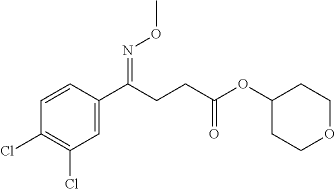 | 4-(3,4-Dichloro-phenyl)-4- methoxyimino-butyric acid tetrahydro-pyran-4-yl ester | 360 |
| 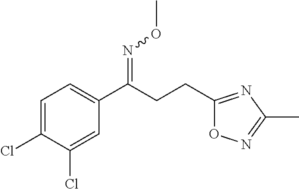 | 1-(3,4-Dichloro-phenyl)-3-(3- methyl-[1,2,4]oxadiazol-5- yl)-propan-1-one O-methyl- oxime | 314 |
| 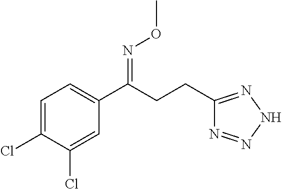 | 1-(3,4-Dichloro-phenyl)-3- (2H-tetrazol-5-yl)-propan-1- one O-methyl-oxime | 300 |
| 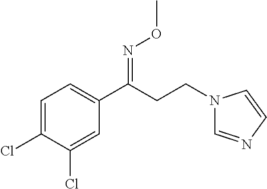 | 1-(3,4-Dichloro-phenyl)-3- imidazol-1-yl-propan-1-one O-methyl-oxime | 298 |
| 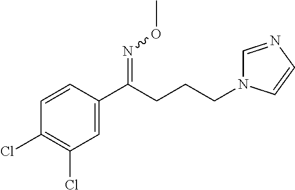 | 1-(3,4-Dichloro-phenyl)-4- imidazol-1-yl-butan-1-one O- methyl-oxime | 312 |
| 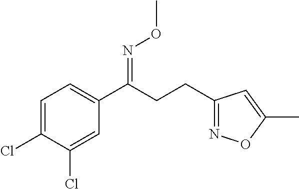 | 1-(3,4-Dichloro-phenyl)-3-(5- methyl-isoxazol-3-yl)- propan-1-one O-methyl- oxime | 313 |
| 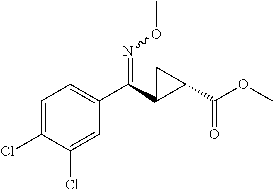 | (1S, 2S)-trans-2-[(3,4- Dichloro-phenyl)- methoxyimino-methyl]- cyclopropanecarboxylic acid methyl ester | 302 |
| 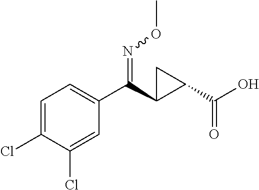 | (1S, 2S)-trans-2-[(3,4- Dichloro-phenyl)- methoxyimino-methyl]- cyclopropanecarboxylic acid | 288 |
| 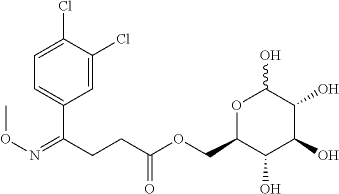 | 4-(3,4-Dichloro-phenyl)-4- methoxyimino-butyric acid 3,4,5,6-tetrahydroxy- tetrahydro-pyran-2-ylmethyl ester | 438 |
| 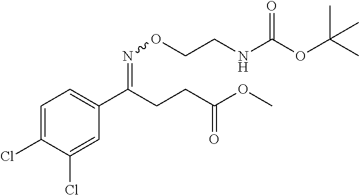 | 4-[2-tert- Butoxycarbonylamino- ethoxyimino]-4-(3,4- dichloro-phenyl)-butyric acid methyl ester | 419/421 |
| 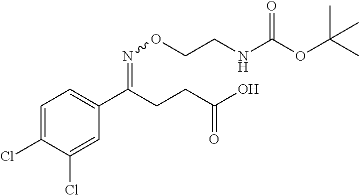 | 4-[2-tert- Butoxycarbonylamino- ethoxyimino]-4-(3,4- dichloro-phenyl)-butyric acid | 405/407 |
| 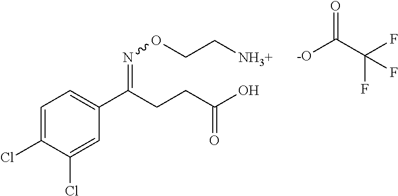 | 4-[2-Amino-ethoxyimino]-4- (3,4-dichloro-phenyl)-butyric acid trifluoroacetic acid salt | 305/307 |
| 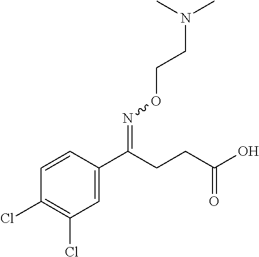 | 4-(3,4-Dichloro-phenyl)-4-(2- dimethylamino-ethoxyimino)- butyric acid | 332/334 |
| 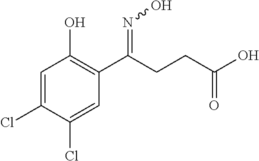 | 4-(4,5-Dichloro-2-hydroxy- phenyl)-4-hydroxyimino- butyric acid | 277/279 |
| 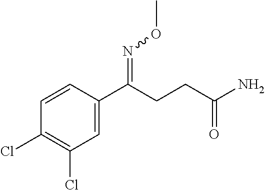 | 4-(3,4-Dichloro-phenyl)-4- methoxyimino-butyramide | 274/276 |
| 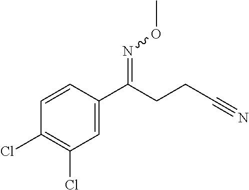 | 4-(3,4-Dichloro-phenyl)-4- methoxyimino-butyronitrile | 256/258 |
| 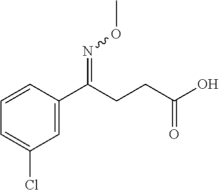 | 4-(3-Chloro-phenyl)-4- methoxyimino-butyric acid | 242 |
| 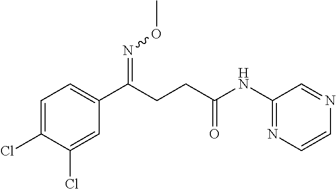 | 4-(3,4-Dichloro-phenyl)-4- methoxyimino-N-pyrazin-2- yl-butyramide | 353 |
| 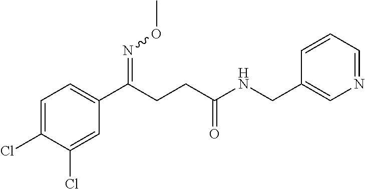 | 4-(3,4-Dichloro-phenyl)-4- methoxyimino-N-pyridin-3- ylmethyl-butyramide | 366 |
| 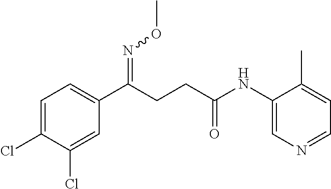 | 4-(3,4-Dichloro-phenyl)-4- methoxyimino-N-(4-methyl- pyridin-3-yl)-butyramide | 366 |
| 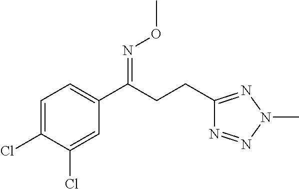 | 1-(3,4-Dichloro-phenyl)-3-(2- methyl-2H-tetrazol-5-yl)- prop-1-one O-methyl- oxime | 313 |
| 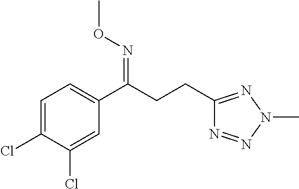 | 1-(3,4-Dichloro-phenyl)-3-(2- methyl-2H-tetrazol-5-yl)- propan-1-one O-methyl- oxime | 313 |
| 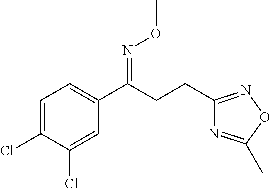 | 1-(3,4-Dichloro-phenyl)-3-(5- methyl-[1,2,4]oxadiazol-3- yl)-propan-1-one O-methyl- oxime | 314/316 |
| 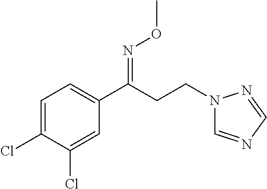 | 1-(3,4-Dichloro-phenyl)-3- [1,2,4]triazol-1-yl-propan-1- one O-methyl-oxime | 299/301 |
| 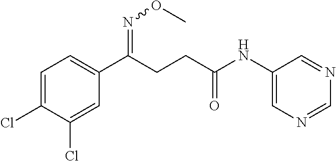 | 4-(3,4-Dichloro-phenyl)-4- methoxyimino-N-pyrimidin- 5-yl-butyramide | 353 |
| 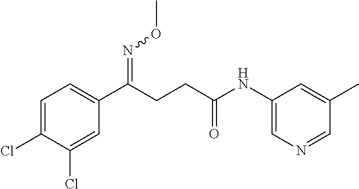 | 4-(3,4-Dichloro-phenyl)-4- methoxyimino-N-(5-methyl- pyridin-3-yl)-butyramide | 366 |
| 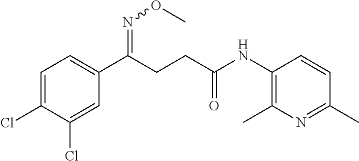 | 4-(3,4-Dichloro-phenyl)-N- (2,6-dimethyl-pyridin-3-yl)- 4-methoxyimino-butyramide | 380 |
| 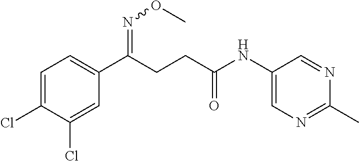 | 4-(3,4-Dichloro-phenyl)-4- methoxyimino-N-(2-methyl- pyrimidin-5-yl)-butyramide | 367 |
| 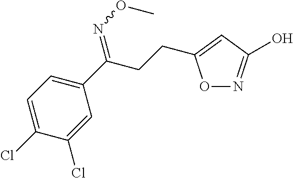 | 1-(3,4-Dichloro-phenyl)-3-(3- hydroxy-isoxazol-5-yl)- propan-1-one O-methyl- oxime | 315 |
| 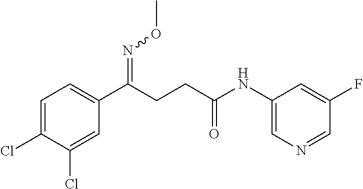 | 4-(3,4-Dichloro-phenyl)-N- (5-fluoro-pyridin-3-yl)-4- methoxyimino-butyramide | 370 |
| 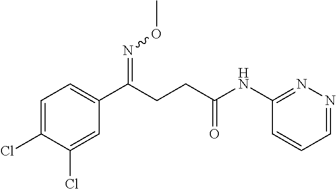 | 4-(3,4-Dichloro-phenyl)-4- methoxyimino-N-pyridazin- 3-yl-butyramide | 353 |
| 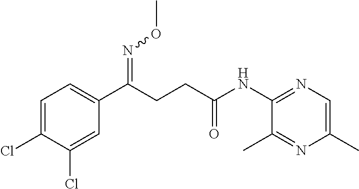 | 4-(3,4-Dichloro-phenyl)-N- (3,5-dimethyl-pyrazin-2-yl)- 4-methoxyimino-butyramide | 381 |
| 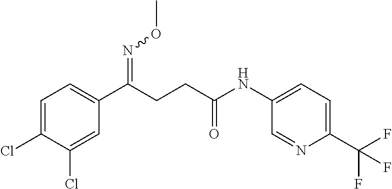 | 4-(3,4-Dichloro-phenyl)-4- methoxyimino-N-(6- trifluoromethyl-pyridin-3-yl)- butyramide | 420 |
| 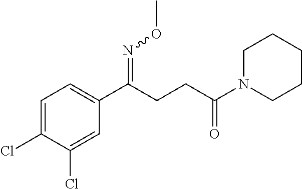 | 1-(3,4-Dichloro-phenyl)-4- piperidin-1-yl-butane-1,4- dione 1-(O-methyl-oxime) | 343 |
| 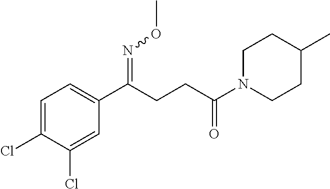 | 1-(3,4-Dichloro-phenyl)-4-(4- methyl-piperidin-1-yl)- butane-1,4-dione 1-(O- methyl-oxime) | 357 |
| 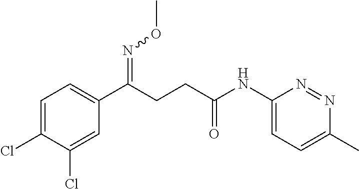 | 4-(3,4-Dichloro-phenyl)-4- methoxyimino-N-(6-methyl- pyridazin-3-yl)-butyramide | 366 |
| 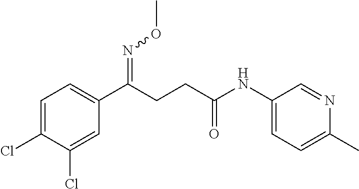 | 4-(3,4-Dichloro-phenyl)-4- methoxyimino-N-(6-methyl- pyridin-3-yl)-butyramide | 365 |
| 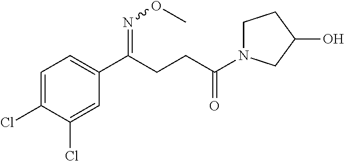 | 1-(3,4-Dichloro-phenyl)-4-(3- hydroxy-pyrrolidin-1-yl)- butane-1,4-dione 1-(O- methyl-oxime) | 345 |
| 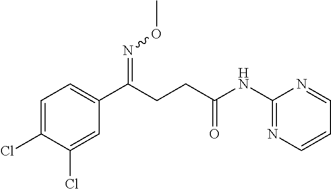 | 4-(3,4-Dichloro-phenyl)-4- methoxyimino-N-pyrimidin- 2-yl-butyramide | 352 |
| 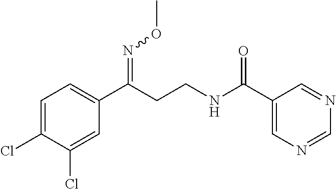 | Pyrimidine-5-carboxylic acid {3-(3,4-dichloro-phenyl)-3- methoxyimino-propyl}-amide | 353 |
| 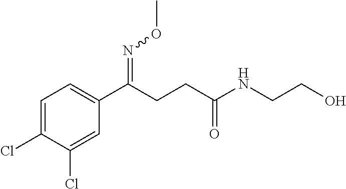 | 4-(3,4-Dichloro-phenyl)-N- (2-hydroxy-ethyl)-4- methoxyimino-butyramide | 319 |
| 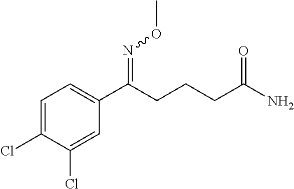 | 5-(3,4-Dichloro-phenyl)-5- methoxyimino-pentanoic acid amide | 289 |
| 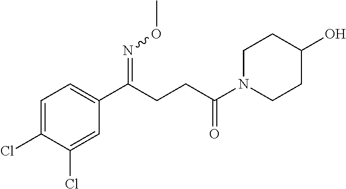 | 1-(3,4-Dichloro-phenyl)-4-(4- hydroxy-piperidin-1-yl)- butane-1,4-dione1-(O- methyl-oxime) | 359 |
| 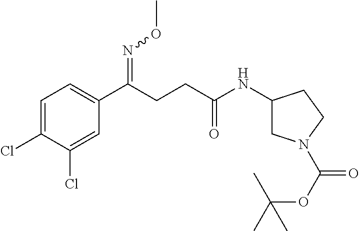 | 3-{4-(3,4-Dichloro-phenyl)- 4-methoxyimino- butyrylamino}-pyrrolidine-1- carboxylic acid tert-butyl ester | [M + H + Na]⁺ = 466 |
| 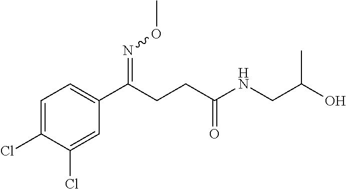 | 4-(3,4-Dichloro-phenyl)-N- (2-hydroxy-propyl)-4- methoxyimino-butyramide | 333 |
| 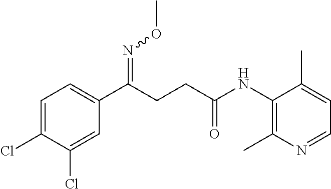 | 4-(3,4-Dichloro-phenyl)-N- (2,4-dimethyl-pyridin-3-yl)- 4-methoxyimino-butyramide | 380 |
| 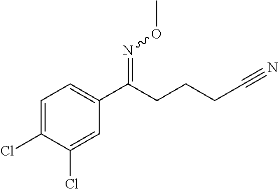 | 5-(3,4-Dichloro-phenyl)-5- methoxyimino-pentanenitrile | 271 |
| 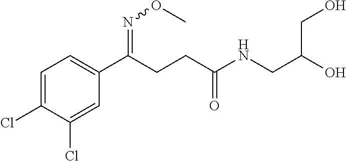 | 4-(3,4-Dichloro-phenyl)-N- (2,3-dihydroxy-propyl)-4-- methoxyimino-butyramide | 349 |
| 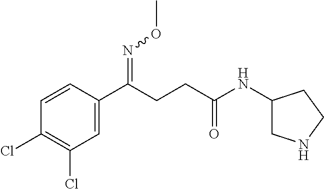 | 4-(3,4-Dichloro-phenyl)-4- methoxyimino-N-pyrrolidin- 3-yl-butyramide | 344 |
| 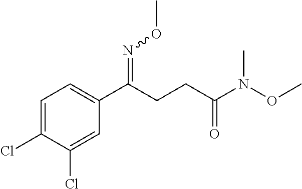 | 4-(3,4-Dichloro-phenyl)-N- methoxy-4-methoxyimino-N- methyl-butyramide | 319 |
| 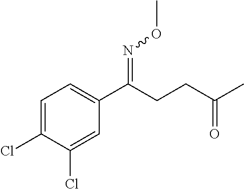 | 1-(3,4-Dichloro-phenyl)- pentane-1,4-dione 1-(O- methyl-oxime) | 274 |
| 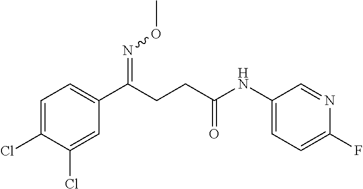 | 4-(3,4-Dichloro-phenyl)-N- (6-fluoro-pyridin-3-yl)-4- methoxyimino-butyramide | 370 |
| 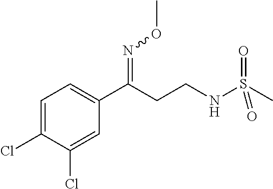 | N-{3-(3,4-Dichloro-phenyl)- 3-methoxyimino-propyl}- methanesulfonamide | 325 |
| 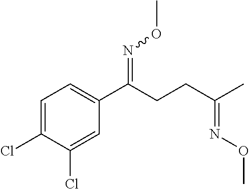 | 1-(3,4-Dichloro-phenyl)- pentane-1,4-dione bis-(O- methyl-oxime) | 303 |
| 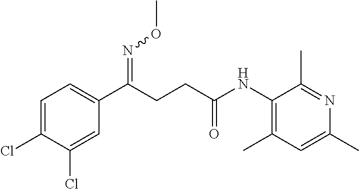 | 4-(3,4-Dichloro-phenyl)-4- methoxyimino-N-(2,4,6- trimethyl-pyridin-3-yl)- butyramide | 394 |
| 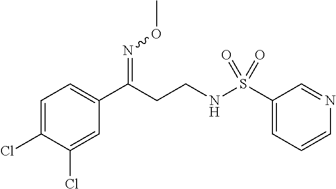 | Pyridine-3-sulfonic acid {3- (3,4-dichloro-phenyl)-3- methoxyimino-propyl}-amide | 388 |
| 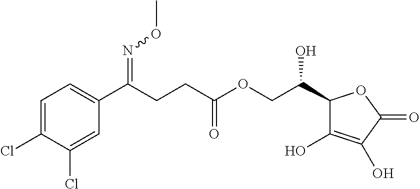 | 4-(3,4-Dichloro-phenyl)-4- methoxyimino-butyric acid (S)-2-((R)-3,4-dihydroxy-5- oxo-2,5-dehydro-furan-2-yl)- 2-hydroxy-ethyl ester | 434 |
| 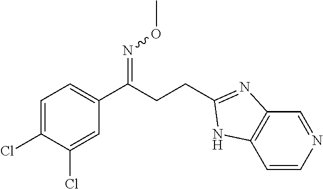 | 1-(3,4-Dichloro-phenyl)-3- (1H-imidazo[4,5-c]pyridin-2- yl)-propan-1-one O-methyl- oxime | 349 |

### Example 14

A generalized procedure for monitoring L-Kynurenine (KYN) hydroxylation to form product 3-Hydroxy-Kynurenine (3OH-KYN) by LC/MS is described below. Product is quantified by multiple reaction monitoring using MS.

### Key Reagents:

Compound: Stock concentrations: 10 mM in 100% DMSO
Cell line: CHO GST HIS KMO cell line, 1E4 cells/well/100 μl in 96 well cell plate
Substrate: L-Kynurenine (Sigma: Cat# K3750, stock concentration: 10 mM in 100 mM potassium phosphate buffer, pH 7.4)

### Assay Conditions:

Medium: OptiMem (Reduced Serum Medium 1×, +L-Glutamine+HEPES−Phenol Red; GIBCO: Cat# 11058)
Assay Volume: 200 μl
Plate Format: 96 well plate, transparent (Corning)
Read-Out: product (3OH-KYN) quantification using product specific MRM
Reader: LC/MS/MS

### Assay Protocol:

prepare serial dilution (factor 3) of compound in 100% DMSO (top concentration=6.67 mM, 100% DMSO)
[8 points: 6.67 mM; 2.22 mM; 0.74 mM; 0.247 mM; 0.082 mM; 0.027 mM; 0.009 mM; 0.003 mM]
prepare 300-fold concentrated solution of each compound concentration (top concentration 22.22 μM, 0.3% DMSO) in OptiMem medium
[22.2 μM; 7.41 μM; 2.47 μM; 0.82 μM; 0.27 μM; 0.09 μM; 0.03 μM; 0.01 μM]
prepare substrate (10 mM) at concentration of 1.1 mM in medium
medium of cell plate is drawed off
cells are washed with OptiMem (100 μl/well) and thawed off again
assay mix: 90 μl OptiMem/well+90 μl compound/well of each concentration
[final compound top concentration: 10 μM; 0.15% DMSO]
[final compound bottom concentration: 0.004 μM; 0.15% DMSO]
pre-incubation: 30 min at 37° C.
add 20 μl/well of the 1.1 mM substrate solution (final assay concentration: 100 μM)
positive control: 200 μl OptiMem
negative control: 180 μl OptiMem+20 μl 1.1 mM substrate
incubate ˜24 h at 37° C.
transfer 100 μl of each well in a transparent 96 well plate (Corning)
add 100 μl/well 10% trichloro acetic acid (TCA) in water
centrifugate plate for 3 min at 4000 rpm
detect product by LC/MS (injection of 50 μl/well; 2.5 fold overfill of the 20 μl sample loop)
   Data analysis: IC₅₀'s are calculated using automated fitting algorithm (A+Analysis).

### Example 15

A method of monitoring L-Kynurenine (KYN) hydroxylation to form product 3-Hydroxy-Kynurenine (3OH-KYN) by LC/MS is described below. Product is quantified by multiple reaction monitoring.

### Key Reagents:

Compound: Stock concentrations: 10 mM in 100% DMSO
Enzyme: KMO enzyme prepared at Evotec via mitochondria isolation from CHO-GST HIS KMO cells
Substrate: L-Kynurenine (Sigma: Cat# K3750)
   [stock concentration: 10 mM in 100 mM potassium phosphate buffer, pH 7.4]

### Assay Conditions:

Buffer: 100 mM potassium phosphate, pH 7.4, 200 μM NADPH, 0.4 U/ml G6P-DH (Glucose 6-phosphate dehydrogenase), 3 mM G6P (D-Glucose 6-phosphate)
Assay Volume: 40 μl
Plate Format: 384 well plate, transparent (Matrix)
Read-Out: product (3OH-KYN) quantification using product specific MRM
Reader: LC/MS/MS

### Assay Protocol:

prepare serial dilution (factor 3) of compound in 100% DMSO (top concentration=10 mM, 100% DMSO)
[8 points: 10 mM; 3.33 mM; 1.11 mM; 0.37 mM; 0.12 mM; 0.04 mM; 0.0137 mM; 0.0045 mM, 0.0015 mM]
prepare 3.33-fold concentrated solution of each compound concentration (top concentration 300 μM, 3% DMSO) in assay buffer
[concentrations: 300 μM; 100 μM; 33.3 μM; 11.1 μM; 3.70 μM; 1.23 μM; 0.41 μM; 0.137 μM]
prepare substrate (10 mM) at concentration of 1 mM in assay buffer
assay mix: 4 μl compound/well of each concentration+24 μl assay buffer/well+8 μl KMO human enzyme+4 μl 1 mM substrate (final concentration=100 μM)
[final compound top concentration: 30 μM; 0.3% DMSO]
[final compound bottom concentration: 0.0137 μM; 0.3% DMSO]
positive control: 4 μl 50 μM FCE28833 in assay buffer [0.5% DMSO] (final assay concentration=5 μM)+24 μl assay buffer/well+8 μl KMO human enzyme+4 μl 1 mM substrate (final concentration=100 μM)
negative control: 28 μl assay buffer/well+8 μl KMO human enzyme+4 μl 1 mM substrate (final concentration=100 μM)
incubate 400 min at RT
add 40 μl/well 10% trichloro acetic acid in water to stop the assay and precipitate protein
centrifuge plate for 3 min at 4000 rpm
product detection by LC/MS (injection of 50 μl/well; 2.5 fold overfill of the 20 μl sample loop)
   Data analysis: IC₅₀'s are calculated using automated fitting algorithm (A+Analysis).

### Example 16

A method of monitoring L-Kynurenine (KYN) hydroxylation to form 3-Hydroxy-Kynurenine (3OH-KYN) by LC/MS is described. Product is quantified by multiple reaction monitoring (MRM method).

### Key Reagents:

Compound: Stock concentrations: 10 mM in 100% DMSO
Enzyme: KMO enzyme prepared at Evotec from mouse liver (4-6 weeks old) via mitochondria isolation as described in the literature
Substrate: L-Kynurenine (Sigma: Cat# K3750, stock concentration: 10 mM in 100 mM potassium phosphate buffer, pH 7.4)

### Assay Conditions:

Buffer: 100 mM potassium phosphate, pH 7.4, 200 μM NADPH, 0.4 U/ml G6P-DH (Glucose 6-phosphate Dehydrogenase), 3 mM G6P (D-Glucose 6-phosphate)
Assay Volume: 40 μl
Plate Format: 384 well plate, transparent (Matrix)
Read-Out: product (3OH-KYN) quantification using product specific MRM
Reader: LC/MS/MS

### Assay Protocol:

prepare serial dilution (factor 3) of compound in 100% DMSO (top concentration=10 mM, 100% DMSO)
[8 points: 10 mM; 3.33 mM; 1.11 mM; 0.37 mM; 0.12 mM; 0.04 mM; 0.0137 mM; 0.0045 mM, 0.0015 mM]
prepare 3.33-fold concentrated solution of each compound concentration (top concentration 300 μM, 3% DMSO) in assay buffer
[concentrations: 300 μM; 100 μM; 33.3 μM; 11.1 μM; 3.70 μM; 1.23 μM; 0.41 μM; 0.137 μM]
prepare substrate (10 mM) at concentration of 1 mM in assay buffer
assay mix: 4 μl compound/well of each concentration+24 μl assaybuffer/well+8 μl KMO mouse enzyme+4 μl 1 mM substrate (final concentration=100 μM)
[final compound top concentration: 30 μM; 0.3% DMSO]
[final compound bottom concentration: 0.0137 μM; 0.3% DMSO]
positive control: 4 μl 50 μM FCE28833 in assay buffer, 0.5% DMSO [final assay concentration=5 μM]+24 μl assaybuffer/well+8 μl KMO mouse enzyme+4 μl 1 mM substrate [final concentration=100 μM]
negative control: 28 μl assay buffer/well+8 μl KMO mouse enzyme+4 μl 1 mM substrate [final concentration=100 μM]
incubate 40 min at RT
add 40 μl/well 10% trichloro acetic acid in water to stop the assay and precipitate protein
centrifuge plate for 3 min at 4000 rpm
product detection by LC/MS (injection of 20 μl/well, 2 fold overfill of the 10 μl sample loop)
   Data analysis: IC₅₀'s are calculated using automated fitting algorithm (A+Analysis).

### Example 17

Using procedures similar to those described herein, the following compounds were assayed for activity.

| Compound | INH.Mouse @ 10 μM |
|---|---|
| 4-(3,4-Dichloro-phenyl)-4-methoxyimino- | 105.94 |
| butyric acid | |
| 4-(3,4-Dichloro-phenyl)-4-ethoxyimino- | 106.65 |
| butyric acid | |
| 4-Benzyloxyimino-4-(3,4-dichloro-phenyl)- | 61.85 |
| butyric acid | |
| 4-Cyclopropylmethoxyimino-4-(3,4- | 99.62 |
| dichloro-phenyl)-butyric acid | |
| 4-(3,5-Dichloro-phenyl)-4-methoxyimino- | 98.74 |
| butyric acid | |
| 4-(3,4-Dichloro-phenyl)-4-methoxyimino- | 105.7 |
| butyric acid methyl ester | |
| 4-(3,4-Dichloro-phenyl)-4-ethoxyimino- | 106.7 |
| butyric acid methyl ester | |
| 4-Benzyloxyimino-4-(3,4-dichloro-phenyl)- | 65.95 |
| butyric acid methyl ester | |
| 4-(3,4-Dichloro-phenyl)-4-hydroxyimino- | 99.39 |
| butyric acid methyl ester | |
| 5-(3,4-Dichloro-phenyl)-5-methoxyimino- | 61.23 |
| pentanoic acid methyl ester | |
| 5-(3,4-Dichloro-phenyl)-5-methoxyimino- | 78.35 |
| pentanoic acid | |
| 4-(3,4-Dichloro-phenyl)-4-methoxyimino-N- | 102.87 |
| phenyl-butyramide | |
| 4-(3,4-Dichloro-phenyl)-4-methoxyimino-N- | 102.78 |
| pyridin-3-yl-butyramide | |
| 4-(3,4-Dichloro-phenyl)-4-methoxyimino-N- | 54.6 |
| (2-morpholin-4-yl-ethyl)-butyramide | |
| 4-(3,4-Dichloro-phenyl)-4-methoxyimino-N- | 95.17 |
| [1,3,4]thiadiazol-2-yl-butyramide | |
| 4-(3,4-Dichloro-phenyl)-4-methoxyimino-N- | 76.45 |
| pyridin-4-yl-butyramide | |
| 4-(3,4-Dichloro-phenyl)-4-methoxyimino-N- | 104.23 |
| pyridin-2-yl-butyramide | |
| 4-(3,4-Dichloro-phenyl)-N-isoxazol-3-yl-4- | 101.15 |
| methoxyimino-butyramide | |
| 4-(3,4-Dichloro-phenyl)-4-methoxyimino-N- | 90.53 |
| (1-methyl-1H-pyrazol-4-yl)-butyramide | |
| 1-(3,4-Dichloro-phenyl)-4-morpholin-4-yl- | 63.43 |
| butane-1,4-dione 1-(O-methyl-oxime) | |
| 4-(3,4-Dichloro-phenyl)-N-ethyl-4- | 98.87 |
| methoxyimino-butyramide | |
| 4-(3,4-Dichloro-phenyl)-N-isopropyl-4- | 94.28 |
| methoxyimino-butyramide | |
| N-Cyclopropyl-4-(3,4-dichloro-phenyl)-4- | 100.2 |
| methoxyimino-butyramide | |
| 4-(3,4-Dichloro-phenyl)-4-methoxyimino-N- | 93.73 |
| methyl-butyramide | |
| 4-[4-(3,4-Dichloro-phenyl)-4- | 75.16 |
| methoxyimino-butyrylamino]-piperidine-1- | |
| carboxylic acid tert-butyl ester | |
| {2-[4-(3,4-Dichloro-phenyl)-4- | 68 |
| methoxyimino-butyrylamino]-ethyl}- | |
| dimethyl-ammonium trifluoroacetate | |
| 4-(3,4-Dichloro-phenyl)-4-methoxyimino- | 64.34 |
| N,N-dimethyl-butyramide | |
| 4-(3,4-Dichloro-phenyl)-4-methoxyimino- | 102.33 |
| butyric acid isopropyl ester | |
| 4-(3,4-Dichloro-phenyl)-4-methoxyimino- | 102.16 |
| butyric acid cyclopentyl ester | |
| 4-(3,4-Dichloro-phenyl)-4-methoxyimino- | 101.88 |
| butyric acid cyclobutyl ester | |
| 4-(3,4-Dichloro-phenyl)-4-methoxyimino- | 104.72 |
| butyric acid pyridin-3-yl ester | |
| 4-(3,4-Dichloro-phenyl)-4-methoxyimino- | 101.85 |
| butyric acid tetrahydro-pyran-4-yl ester | |
| 1-(3,4-Dichloro-phenyl)-3-(2H-tetrazol-5- | 96.39 |
| yl)-propan-1-one O-methyl-oxime | |
| (1S,2S)-trans-2-[(3,4-Dichloro-phenyl)- | 60.95 |
| methoxyimino-methyl]- | |
| cyclopropanecarboxylic acid methyl ester | |
| (1S,2S)-trans-2-[(3,4-Dichloro-phenyl)- | 81.18 |
| methoxyimino-methyl]- | |
| cyclopropanecarboxylic acid | |
| 4-[2-tert-Butoxycarbonylamino- | 56.37 |
| ethoxyimino]-4-(3,4-dichloro-phenyl)- | |
| butyric acid methyl ester | |
| 4-[2-tert-Butoxycarbonylamino- | 61.86 |
| ethoxyimino]-4-(3,4-dichloro-phenyl)- | |
| butyric acid | |
| 4-[2-Amino-ethoxyimino]-4-(3,4-dichloro- | 49.52 |
| phenyl)-butyric acid trifluoroacetic acid salt | |
| 4-(3,4-Dichloro-phenyl)-4-methoxyimino- | 94.92 |
| butyramide | |
| 4-(3,4-Dichloro-phenyl)-4-methoxyimino- | 74.63 |
| butyronitrile | |
| 4-(3-Chloro-phenyl)-4-methoxyimino- | 101.48 |
| butyric acid | |
| 4-(3,4-Dichloro-phenyl)-4-methoxyimino-N- | 97.97 |
| pyrazin-2-yl-butyramide | |
| 4-(3,4-Dichloro-phenyl)-4-methoxyimino-N- | 77.08 |
| pyridin-3-ylmethyl-butyramide | |
| 4-(3,4-Dichloro-phenyl)-4-methoxyimino-N- | 93.9 |
| (4-methyl-pyridin-3-yl)-butyramide | |
| 1-(3,4-Dichloro-phenyl)-3-(5-methyl- | 87.22 |
| [1,2,4]oxadiazol-3-yl)-propan-1-one O- | |
| methyl-oxime | |
| 4-(3,4-Dichloro-phenyl)-4-methoxyimino-N- | 98.52 |
| pyrimidin-5-yl-butyramide | |
| 4-(3,4-Dichloro-phenyl)-4-methoxyimino-N- | 102.25 |
| (5-methyl-pyridin-3-yl)-butyramide | |
| 4-(3,4-Dichloro-phenyl)-N-(2,6-dimethyl- | 93.01 |
| pyridin-3-yl)-4-methoxyimino-butyramide | |
| 4-(3,4-Dichloro-phenyl)-4-methoxyimino-N- | 90.39 |
| (2-methyl-pyrimidin-5-yl)-butyramide | |
| 4-(3,4-Dichloro-phenyl)-N-(5-fluoro- | 90.33 |
| pyridin-3-yl)-4-methoxyimino-butyramide | |
| 4-(3,4-Dichloro-phenyl)-4-methoxyimino-N- | 101.34 |
| pyridazin-3-yl-butyramide | |
| 4-(3,4-Dichloro-phenyl)-N-(3,5-dimethyl- | 101.21 |
| pyrazin-2-yl)-4-methoxyimino-butyramide | |
| 1-(3,4-Dichloro-phenyl)-4-piperidin-1-yl- | 60.43 |
| butane-1,4-dione 1-(O-methyl-oxime) | |
| 1-(3,4-Dichloro-phenyl)-4-(4-methyl- | 57.27 |
| piperidin-1-yl)-butane-1,4-dione 1-(O- | |
| methyl-oxime) | |
| 4-(3,4-Dichloro-phenyl)-4-methoxyimino-N- | 90.25 |
| (6-methyl-pyridazin-3-yl)-butyramide | |
| 4-(3,4-Dichloro-phenyl)-4-methoxyimino-N- | 82.89 |
| (6-methyl-pyridin-3-yl)-butyramide | |
| 4-(3,4-Dichloro-phenyl)-4-methoxyimino-N- | 101.42 |
| pyrimidin-2-yl-butyramide | |
| 4-(3,4-Dichloro-phenyl)-N-(2-hydroxy- | 95.65 |
| ethyl)-4-methoxyimino-butyramide | |
| 4-(3,4-Dichloro-phenyl)-N-(2-hydroxy- | 98.83 |
| propyl)-4-methoxyimino-butyramide | |
| 4-(3,4-Dichloro-phenyl)-N-(2,4-dimethyl- | 85.05 |
| pyridin-3-yl)-4-methoxyimino-butyramide | |
| 4-(3,4-Dichloro-phenyl)-4-methoxyimino-N- | 62.52 |
| methyl-N-pyridin-3-yl-butyramide | |
| 5-(3,4-Dichloro-phenyl)-5-methoxyimino- | 71.5 |
| pentanenitrile | |
| 4-(3,4-Dichloro-phenyl)-N-(2,3-dihydroxy- | 75.7 |
| propyl)-4-methoxyimino-butyramide | |
| 4-(3,4-Dichloro-phenyl)-4-methoxyimino-N- | 74.27 |
| pyrrolidin-3-yl-butyramide | |
| 4-(3,4-Dichloro-phenyl)-N-methoxy-4- | 101.75 |
| methoxyimino-N-methyl-butyramide | |
| 1-(3,4-Dichloro-phenyl)-pentane-1,4-dione | 77.73 |
| 1-(O-methyl-oxime) | |
| 4-(3,4-Dichloro-phenyl)-N-(6-fluoro- | 91.69 |
| pyridin-3-yl)-4-methoxyimino-butyramide | |
| 4-(3,4-Dichloro-phenyl)-4-methoxyimino-N- | 66.28 |
| (2,4,6-trimethyl-pyridin-3-yl)-butyramide | |
| 4-(3,4-Dichloro-phenyl)-4-methoxyimino- | 101.97 |
| butyric acid (S)-2-((R)-3,4-dihydroxy-5-oxo- | |
| 2,5-dihydro-furan-2-yl)-2-hydroxy-ethyl | |
| ester | |
| 1-(3,4-Dichloro-phenyl)-3-(1H-imidazo[4,5- | 64.33 |
| c]pyridin-2-yl)-propan-1-one O-methyl- | |
| oxime | |

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

1. At least one chemical entity chosen from compounds of formula I 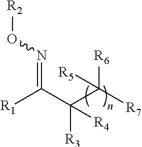
and pharmaceutically acceptable salts and prodrugs thereof wherein:
R₁ is chosen from aryl and heteroaryl, each of which is substituted with one, two, or three groups chosen from halo, lower alkyl, alkoxy, and hydroxy;
R₂ is chosen from hydrogen and optionally substituted lower alkyl;
R₃ and R₄ are independently chosen from hydrogen, halo, hydroxy, lower alkyl, and lower alkoxy;
for each occurrence, R₅ and R₆ are independently chosen from hydrogen and lower alkyl;
or R₃ and R₅, taken together with the atoms to which they are attached, form an optionally substituted cycloalkyl ring,
n is one or two;
R₇ is chosen from —C(O)OR₈, —C(O)R₈, optionally substituted amino, —C(═N—OR₁₁)R₈, —C(O)NR₉R₁₀, cyano, and optionally substituted heteroaryl;
R₈ is chosen from hydrogen, optionally substituted lower alkyl, optionally substituted aryl, optionally substituted heteroaryl, optionally substituted cycloalkyl, optionally substituted heterocycloalkyl, and glycosyl; and
R₉ and R₁₀ are independently chosen from hydrogen, optionally substituted lower alkyl, optionally substituted lower alkoxy, optionally substituted cycloalkyl, optionally substituted aryl, optionally substituted heterocycloalkyl, and optionally substituted heteroaryl;
or R₉ and R₁₀, together with the nitrogen to which they are bound, form an optionally substituted heterocycloalkyl or optionally substituted heteroaryl ring;
R₁₁ is chosen from hydrogen and optionally substituted lower alkyl,
provided that if R₁ is 4-bromophenyl, 4-fluorophenyl, 4-methoxyphenyl, 4-methylphenyl, 3-methoxyphenyl, 3-chloro-4-n-propoxyphenyl, 3-chloro-4-ethoxyphenyl, 3-chloro-4-n-butoxyphenyl, 2,3,4-trimethylphenyl, 3,4,5-trimethylphenyl, 3-chloro-4-methoxyphenyl, 2,4-dimethoxyphenyl, or 2-hydroxy-5-methylphenyl;
n is 1;
R₂, R₃, R₄, R₅, and R₆ are hydrogen; and
R₇ is —C(O)OR₈, then R₈ is not methyl, ethyl, or hydrogen;
provided that if R₁ is 4-fluorophenyl;
n is 1;
R₂, R₃, R₄ and R₅ are hydrogen;
R₆ is methyl; and
R₇ is —C(O)OR₈, then R₈ is not ethyl;
provided that if R₁ is 4-chlorophenyl;
n is 1;
R₂, R₃, R₄ and R₅ are hydrogen and R₆ is methyl, ethyl, or butyl; or
R₂, R₃, and R₄ and R₅ and R₆ are ethyl; and
R₇ is —C(O)OR₈, then R₈ is not ethyl;
provided that if R₁ is 5-bromo-2-methoxyphenyl;
n is 2;
R₂, R₃, R₄, R⁵, and R₆ are hydrogen; and
R₇ is —C(O)OR₈, then R₈ is not methyl or ethyl;
provided that if R₁ is 2-hydroxyphenyl, 4-hydroxyphenyl, 2,4-dihydroxy-6-methylphenyl, 4-ethoxyphenyl, or 4-methoxyphenyl;
n is 2;
R₂, R₃, R₄, R₅, and R₆ are each hydrogen; and
R₇ is —C(O)OR₈, then R₈ is not hydrogen;
provided that if R₁ is 3-chloro-4-methoxyphenyl or 2,5-dimethylphenyl;
n is 1;
R₂, R₃, R₄, and R₅ are hydrogen;
R₆ is ethyl or propyl; and
R₇ is —C(O)OR₈, then R₈ is not hydrogen;
provided that if R₁ is 4-methylphenyl;
n is 1;
R₂, R₃, R₄ and R₅ are hydrogen;
R₆ is methyl; and
R₇ is —C(O)OR₈, then R₈ is not hydrogen; and
provided that if R₁ is 4-methoxyphenyl;
n is 1;
R₂, R₄, R₅ and R₆ are hydrogen;
R₃ is methyl; and
R₇ is —C(O)OR₈, then R₈ is not hydrogen.

2. At least one chemical entity of claim 1 wherein R₁ is chosen from optionally substituted phenyl and optionally substituted heteroaryl.

3. At least one chemical entity of claim 2 wherein R₁ is chosen from pyridinyl and phenyl substituted with one, two or three halo groups.

4. At least one chemical entity of claim 3 wherein R₁ is phenyl substituted with one or two halo groups.

5. At least one chemical entity of claim 1 wherein R₂ is chosen from hydrogen, lower alkyl and lower alkyl substituted with one to three substituents independently chosen from optionally substituted amino, optionally substituted aryl, and optionally substituted cycloalkyl.

6. At least one chemical entity of claim 5 wherein R₂ is methyl.

7. At least one chemical entity of claim 1 wherein at least one of R₃ and R₄ is hydrogen.

8. At least one chemical entity of claim 7 wherein R₃ and R₄ are hydrogen.

9. At least one chemical entity of claim 1 wherein, for each occurrence, at least one of R₅ and R₆ is hydrogen.

10. At least one chemical entity of claim 9 wherein, for each occurrence, R₅ and R₆ are hydrogen.

11. At least one chemical entity of claim 1 wherein n is one.

12. At least one chemical entity of claim 1 wherein n is two.

13. At least one chemical entity of claim 1 wherein R₃ and R₅, taken together with the atoms to which they are attached, form an optionally substituted cyclopropyl ring.

14. At least one chemical entity of claim 1 wherein R₇ is —C(O)OR₈.

15. At least one chemical entity of claim 14 wherein R₈ is chosen from hydrogen and lower alkyl.

16. At least one chemical entity of claim 15 wherein R₈ is chosen from hydrogen and methyl.

17. At least one chemical entity of claim 1 wherein R₇ is —C(O)NR₉R₁₀.

18. At least one chemical entity of claim 17 wherein R₉ and R₁₀ are independently chosen from hydrogen, optionally substituted lower alkyl, optionally substituted cycloalkyl, optionally substituted aryl, optionally substituted heterocycloalkyl, and optionally substituted heteroaryl.

19. At least one chemical entity of claim 18 wherein R₉ is hydrogen or lower alkyl.

20. At least one chemical entity of claim 19 wherein R₉ is hydrogen.

21. At least one chemical entity of claim 17 wherein R₁₀ is chosen from optionally substituted lower alkyl, optionally substituted cycloalkyl, optionally substituted aryl, optionally substituted heterocycloalkyl, and optionally substituted heteroaryl.

22. At least one chemical entity of claim 21 wherein R₁₀ is chosen from optionally substituted thiadiazolyl, optionally substituted oxadiazolyl, optionally substituted tetrazolyl, optionally substituted pyrrolidinyl, optionally substituted pyrimidinyl, optionally substituted pyridinyl, optionally substituted pyridazinyl, optionally substituted pyrazinyl, and optionally substituted oxazolyl.

23. At least one chemical entity of claim 17 wherein R₉ and R₁₀, together with the nitrogen to which they are bound, form an optionally substituted heterocycloalkyl ring.

24. At least one chemical entity of claim 23 wherein R₉ and R₁₀, together with the nitrogen to which they are bound, form an optionally substituted pyrrolidinyl, optionally substituted morpholinyl, optionally substituted piperazinyl, or optionally substituted piperidinyl ring.

25. At least one chemical entity of claim 1 wherein R₇ is chosen from optionally substituted imidazopyridinyl, optionally substituted indazoyl, optionally substituted oxadiazolyl, optionally substituted triazolyl, optionally substituted isoxazolyl, and optionally substituted tetrazolyl.

26. At least one chemical entity of claim 1 wherein R₇ is —C(═N—OR₁₁))R₈, wherein R₁₁ is lower alkyl and R₈ is lower alkyl.

27. At least one chemical entity of claim 1 wherein the compound of formula I is chosen from
4-(3,4-Dichloro-phenyl)-4-methoxyimino-butyric acid;
4-(3,4-Dichloro-phenyl)-4-ethoxyimino-butyric acid;
4-Benzyloxyimino-4-(3,4-dichloro-phenyl)-butyric acid;
4-Cyclopropylmethoxyimino-4-(3,4-dichloro-phenyl)-butyric acid;
4-(3,5-Dichloro-phenyl)-4-methoxyimino-butyric acid;
4-(3,4-Dichloro-phenyl)-4-methoxyimino-2-methyl-butyric acid;
4-(3,4-Dichloro-phenyl)-4-methoxyimino-3-methyl-butyric acid;
4-Methoxyimino-4-pyridin-2-yl-butyric acid;
4-Methoxyimino-4-pyridin-3-yl-butyric acid;
4-(3,4-Dichloro-phenyl)-4-methoxyimino-butyric acid methyl ester;
4-(3,4-Dichloro-phenyl)-4-ethoxyimino-butyric acid methyl ester;
4-Benzyloxyimino-4-(3,4-dichloro-phenyl)-butyric acid methyl ester;
4-(3,4-Dichloro-phenyl)-4-hydroxyimino-butyric acid methyl ester;
5-(3,4-Dichloro-phenyl)-5-methoxyimino-pentanoic acid methyl ester;
5-(3,4-Dichloro-phenyl)-5-methoxyimino-pentanoic acid;
1-(3,4-Dichloro-phenyl)-5-methoxy-pentan-1-one O-methyl-oxime;
4-(3,4-Dichloro-phenyl)-4-methoxyimino-N-phenyl-butyramide;
4-(3,4-Dichloro-phenyl)-4-methoxyimino-N-pyridin-3-yl-butyramide;
4-(3,4-Dichloro-phenyl)-4-methoxyimino-N-(2-morpholin-4-yl-ethyl)-butyramide;
4-(3,4-Dichloro-phenyl)-4-methoxyimino-N-[1,3,4]thiadiazo1-2-ylbutyramide;
4-(3,4-Dichloro-phenyl)-4-methoxyimino-N-pyridin-4-yl-butyramide;
4-(3,4-Dichloro-phenyl)-4-methoxyimino-N-pyridin-2-yl-butyramide;
4-(3,4-Dichloro-phenyl)-N-isoxazol-3-yl-4-methoxyimino-butyramide;
4-(3,4-Dichloro-phenyl)-4-methoxyimino-N-(1-methyl-1H-pyrazol-4-yl)butyramide;
1-(3,4-Dichloro-phenyl)-4-morpholin-4-yl-butane-1,4-dione 1-(O-methyloxime);
4-(3,4-Dichloro-phenyl)-N-ethyl-4-methoxyimino-butyramide;
4-(3,4-Dichloro-phenyl)-N-isopropyl-4-methoxyimino-butyramide;
N-Cyclopropyl-4-(3,4-dichloro-phenyl)-4-methoxyimino-butyramide;
4-(3,4-Dichloro-phenyl)-4-methoxyimino-N-methyl-butyramide;
4-[4-(3,4-Dichloro-phenyl)-4-methoxyimino-butyrylamino]-piperidine-1carboxylic acid tert-butyl ester;
4-[4-(3,4-Dichloro-phenyl)-4-methoxyimino-butyrylamino]-piperidiniwn trifluoroacetate;
{2-[4-(3,4-Dichloro-phenyl)-4-methoxyimino-butyrylamino]-ethyl}-dimethylammoniwn trifluoroacetate;
4-(3,4-Dichloro-phenyl)-4-methoxyimino-N,N-dimethyl-butyramide;
4-(3,4-Dichloro-phenyl)-4-methoxyimino-2-methyl-N-phenyl-butyramide;
4-(3,4-Dichloro-phenyl)-4-methoxyimino-2-methyl-N-pyridin-3-ylbutyramide;
4-(3,4-Dichloro-phenyl)-4-methoxyimino-butyric acid isopropyl ester;
4-(3,4-Dichloro-phenyl)-4-methoxyimino-butyric acid cyclopentyl ester;
4-(3,4-Dichloro-phenyl)-4-methoxyimino-butyric acid cyclobutyl ester;
4-(3,4-Dichloro-phenyl)-4-methoxyimino-butyric acid pyridin-3-yl ester;
4-(3,4-Dichloro-phenyl)-4-methoxyimino-butyric acid tetrahydro-pyran-4-yl ester;
1-(3,4-Dichloro-phenyl)-3-(3-methyl-[1,2,4]oxadiazol-5-yl)-propan-1-one O-methyl-oxime;
1-(3,4-Dichloro-phenyl)-3-(2H-tetrazol-5-yl)-propan-1-one O-methyl-oxime;
1-(3,4-Dichloro-phenyl)-3-imidazol-1-yl-propan-1-one O-methyl-oxime;
1-(3,4-Dichloro-phenyl)-4-imidazol-1-yl-butan-1-one O-methyl-oxime;
1-(3,4-Dichloro-phenyl)-3-(5-methyl-isoxazol-3-yl)-propan-1-one O-methyloxime;
(1S,2S)-trans-2-[(3,4-Dichloro-phenyl)-methoxyimino-methyl]cyclopropanecarboxylic acid methyl ester;
(1S,2S)-trans-2-[(3,4-Dichloro-phenyl)-methoxyimino-methyl]cyclopropanecarboxylic acid;
4-(3,4-Dichloro-phenyl)-4-methoxyimino-butyric acid 3,4,5,6-tetrahydroxytetrahydro-pyran-2-ylmethyl ester;
4-[2-tert-Butoxycarbonylamino-ethoxyimino]-4-(3,4-dichloro-phenyl)-butyric acid;
4-[2-Amino-ethoxyimino]-4-(3,4-dichloro-phenyl)-butyric acid;
4-(3,4-Dichloro-phenyl)-4-(2-dimethylamino-ethoxyimino)-butyric acid;
4-(4,5-Dichloro-2-hydroxy-phenyl)-4-hydroxyimino-butyric acid;
4-(3,4-Dichloro-phenyl)-4-methoxyimino-butyramide;
4-(3,4-Dichloro-phenyl)-4-methoxyimino-butyronitrile;
4-(3-Chloro-phenyl)-4-methoxyimino-butyric acid;
4-(3,4-Dichloro-phenyl)-4-methoxyimino-N-pyrazin-2-yl-butyramide;
4-(3,4-Dichloro-phenyl)-4-methoxyimino-N-pyridin-3-ylmethyl-butyramide;
4-(3,4-Dichloro-phenyl)-4-methoxyimino-N-(4-methyl-pyridin-3-yl)butyramide;
1-(3,4-Dichloro-phenyl)-3-(2-methyl-2H-tetrazol-5-yl)-propan-1-one O-methyl-oxime;
1-(3,4-Dichloro-phenyl)-3-(2-methyl-2H-tetrazol-5-yl)-propan-1-one O-methyl-oxime;
1-(3,4-Dichloro-phenyl)-3-(5-methyl-[1,2,4]oxadiazol-3-yl)-propan-1-one O-methyl-oxime;
1-(3,4-Dichloro-phenyl)-3-[1,2,4]triazol-1-yl-propan-1-one O-methyl-oxime;
4-(3,4-Dichloro-phenyl)-4-methoxyimino-N-pyrimidin-5-yl-butyramide;
4-(3,4-Dichloro-phenyl)-4-methoxyimino-N-(5-methyl-pyridin-3-yl)-butyramide;
4-(3,4-Dichloro-phenyl)-N-(2,6-dimethyl-pyridin-3-yl)-4-methoxyiminobutyramide;
4-(3,4-Dichloro-phenyl)-4-methoxyimino-N-(2-methyl-pyrimidin-5-yl)butyramide;
1-(3,4-Dichloro-phenyl)-3-(3-hydroxy-isoxazol-5-yl)-propan-1-one O-methyloxime;
4-(3,4-Dichloro-phenyl)-N-(5-fluoro-pyridin-3-yl)-4-methoxyiminobutyramide;
4-(3,4-Dichloro-phenyl)-4-methoxyimino-N-pyridazin-3-yl-butyramide;
4-(3,4-Dichloro-phenyl)-N-(3,5-dimethyl-pyrazin-2-yl)-4-methoxyiminobutyramide;
4-(3,4-Dichloro-phenyl)-4-methoxyimino-N-(6-trifluoromethyl-pyridin-3-yl)butyramide;
1-(3,4-Dichloro-phenyl)-4-piperidin-1-yl-butane-1,4-dione 1-(O-methyloxime);
1-(3,4-Dichloro-phenyl)-4-(4-methyl-piperidin-1-yl)-butane-1,4-dione 1-(O-methyl-oxime);
4-(3,4-Dichloro-phenyl)-4-methoxyimino-N-(6-methyl-pyridazin-3-yl)butyramide;
4-(3,4-Dichloro-phenyl)-4-methoxyimino-N-(6-methyl-pyridin-3-yl)butyramide;
1-(3,4-Dichloro-phenyl)-4-(3-hydroxy-pyrrolidin-1-yl)-butane-1,4-dione 1-(Omethyl-oxime);
4-(3,4-Dichloro-phenyl)-4-methoxyimino-N-pyrimidin-2-yl-butyramide;
Pyrimidine-5-carboxylic acid {3-(3,4-dichloro-phenyl)-3-methoxyiminopropyl}-amide;
4-(3,4-Dichloro-phenyl)-N-(2-hydroxy-ethyl)-4-methoxyimino-butyramide;
5-(3,4-Dichloro-phenyl)-5-methoxyimino-pentanoic acid amide;
1-(3,4-Dichloro-phenyl)-4-(4-hydroxy-piperidin-1-yl)-butane-1,4-dione1-(O-methyl-oxime);
3-[4-(3,4-Dichloro-phenyl)-4-methoxyimino-butyrylamino]-pyrrolidine-1carboxylic acid tert-butyl ester;
4-(3,4-Dichloro-phenyl)-N-(2-hydroxy-propyl)-4-methoxyimino-butyramide;
4-(3,4-Dichloro-phenyl)-N-(2,4-dimethyl-pyridin-3-yl)-4-methoxyiminobutyramide;
5-(3,4-Dichloro-phenyl)-5-methoxyimino-pentanenitrile;
4-(3,4-Dichloro-phenyl)-N-(2,3-dihydroxy-propyl)-4-methoxyimino-butyramide;
4-(3,4-Dichloro-phenyl)-4-methoxyimino-N-pyrrolidin-3-yl-butyramide;
4-(3,4-Dichloro-phenyl)-N-methoxy-4-methoxyimino-N-methyl-butyramide;
1-(3,4-Dichloro-phenyl)-pentane-1,4-dione 1-(O-methyl-oxime);
4-(3,4-Dichloro-phenyl)-N-(6-fluoro-pyridin-3-yl)-4-methoxyimino-butyramide;
N-{3-(3,4-Dichloro-phenyl)-3-methoxyimino-propyl}-methanesulfonamide;
1-(3,4-Dichloro-phenyl)-pentane-1,4-dione bis-(O-methyl-oxime);
4-(3,4-Dichloro-phenyl)-4-methoxyimino-N-(2,4,6-trimethyl-pyridin-3-yl)-butyramide;
Pyridine-3-sulfonic acid {3-(3,4-dichloro-phenyl)-3-methoxyimino-propyl}-amide;
4-(3,4-Dichloro-phenyl)-4-methoxyimino-butyric acid (S)-2-(R)-3,4-dihydroxy-5-oxo-2,5-dihydro-furan-2-yl)-2-hydroxy-ethyl ester; and
1-(3,4-Dichloro-phenyl)-3-(1H-imidazo[4,5-c]pyridin-2-yl)-propan-1-one Omethyl-oxime.

28. A pharmaceutical composition comprising at least one chemical entity of claim 1 and at least one pharmaceutically acceptable excipient.

29. A method of treating a condition or disorder mediated by Kynurenine 3-monooxygenase activity in a subject in need of such a treatment which method comprises administering to the subject a therapeutically effective amount of at least one chemical entity of claim 1.

30. The method of claim 29 wherein at least one chemical entity binds Kynurenine 3-mono-oxygenase.

31. The method of claim 30 wherein said condition or disorder involves a neurodegenerative pathology.

32. The method of claim 31 wherein the neurodegenerative pathology is selected from Huntington's disease, Alzheimer's disease, Parkinson's disease, olivoponto cerebellar atrophy, non-Alzheimer's dementia, multi-infarctual dementia, cerebral amyotrophic lateral sclerosis, cerebral ischemia, cerebral hypoxia, spinal or head trauma and epilepsy.

33. A packaged pharmaceutical composition comprising at least one pharmaceutical composition according claim 28 and instructions for using the composition to treat a subject suffering from a condition or disorder mediated by Kynurenine 3-mono-oxygenase activity.

34. The packaged pharmaceutical composition according to claim 33, wherein the condition or disorder is Huntington's disease.
